(19) United States
(12) Patent Application Publication
(10) Pub. No.: US 2013/0060234 A1
(43) Pub. Date: Mar. 7, 2013

(54) INTUSSUSCEPTING BALLOON CATHETER AND METHODS FOR CONSTRUCTING AND USING THEREOF

(71) Applicants: Doron Besser, Tel Aviv (IL); Eran Harari, Maagan Michael (IL)

(72) Inventors: Doron Besser, Tel Aviv (IL); Eran Harari, Maagan Michael (IL)

(21) Appl. No.: 13/520,345

(22) PCT Filed: Jan. 3, 2010
(86) PCT No.: PCT/IL10/00002

§ 371 Date: Nov. 11, 2012

(51) Int. Cl. A61M 25/10; B23P 17/04; A61M 25/09

(52) U.S. Cl. 604/509; 604/103.04; 604/99.02; 29/428

(57) ABSTRACT

A catheter including an outer conduit and an inner conduit movably disposed therein. The inner conduit includes at least one movable part. The proximal end of the inner conduit is angled piercing the wall of the outer conduit. The catheter includes an inflatable intussusceptable balloon having a proximal margin attached to the distal tip of the outer conduit and a distal margin attached to a portion of the inner conduit extending beyond the distal tip of the outer conduit. The diameter of a first portion of the balloon is larger than the diameter of other portions thereof. The catheter includes means for axially moving the inner conduit within the outer conduit, means for introducing expansion fluid into the balloon and means for permitting axial movement of the inner conduit within the outer conduit, unhindered by the passage of the angled proximal part of the inner conduit through the outer conduit.

### FIELD OF THE INVENTION

This invention relates in general to the fields of medical balloon catheters and more particularly to rapid exchange (monorail) systems and catheters having inflatable intussusceptible stepped balloons and methods of their construction and use.

### BACKGROUND OF THE INVENTION

Catheters are used in various interventional procedures for delivering therapeutic means to a treated site (e.g., body organ or passageway such as blood vessels). In many cases, a catheter with a small distal inflatable balloon is guided to the treated site. Once the balloon is in place it is inflated by the operator for affixing it in place, for expanding a blocked vessel, for placing treatment means (e.g., stent) and/or for delivering surgical tools (e.g. knives, drills etc.) to a desired site. In addition, catheter systems have also been designed and used for retrieval of objects such as stents from body passageways.

Two basic types of catheter have been developed for intravascular use: over-the-wire (OTW) catheters and rapid-exchange (RE) catheters.

OTW catheter systems are characterized by the presence of a full-length guide-wire, such that when the catheter is in its in situ working position, said guide-wire passes through the entire length of a lumen formed in, or externally attached to, the catheter. OTW systems have several operational advantages which are related to the use of a full length guide-wire, including good stiffness and pushability, features which are important when maneuvering balloon catheters along tortuous and/or partially occluded blood vessels.

U.S. Pat. No. 6,039,721 to Johnson et al. describes a balloon catheter system comprising two concentrically-arranged conduits, with a balloon connected between the distal regions thereof. The catheter system permits both expansion/deflation of the balloon and alteration in the length of the balloon when in situ, such that the balloon may be moved between extended and intussuscepted conformations. The catheter system is constructed in order that it may be use for two main purposes: firstly, treatment (i.e. expansion) of different-length stenosed portions of blood vessels with a single balloon and secondly, the delivery of either stents or medication to intravascular lesions, wherein the stent or medication is contained within the distally-intussuscepted portion of the balloon. When used for multiple, differing-length lesion expansion, the balloon is inserted into blood vessel in a collapsed, shortened, intussuscepted conformation, and is advanced until it comes to rest in the region of the shortest lesion to be treated. The balloon is then inflated and the lesion treated (i.e. expanded). Following deflation of the balloon, the distal end of the catheter system is moved such that the balloon becomes positioned in the region of the next-shortest lesion to be treated. The effective length of the balloon is then increased by moving the inner conduit in relation to the proximal conduit, following which the balloon is again inflated and the lesion treated. In this way, a series of different length stenoses, in order from the shortest to the longest, may be treated using a single balloon. When used for stent delivery, the stent is pre-loaded into a proximal annular space formed as a result of balloon intussusception. The balloon is then moved to the desired site and the stent delivered by means of moving the inner conduit distally (in relation to the outer tube), thereby “unpeeling” the stent from the catheter.

WO 2000/38776 discloses a dual-conduit balloon catheter system similar in basic design to that described above in relation to U.S. Pat. No. 6,039,721. This catheter system is intended for use in a vibratory mode in order to break through total occlusions of the vascular lumen. In order to fulfill this aim, the outer conduit has a variable stiffness along its length, while the inner conduit. In addition, the inner conduit while being intrinsically relatively flexible is stiffened by the presence of axial tensioning wires. These conduit design features are used in order to permit optimal translation of vibratory movements of the proximal end of the inner conduit into corresponding vibration of the distal tip thereof.

Rapid exchange (“monorail”) catheters typically comprise a relatively short guide-wire lumen provided in a distal section thereof, and a proximal guide-wire exit port located between the catheter's distal and proximal ends. This arrangement allows exchange of the catheter over a relatively short guide-wire, in a manner which is simple to perform and which can be carried out by a single operator. Rapid exchange catheters have been extensively described in the art, for example, U.S. Pat. Nos. 4,762,129, 4,748,982 and EP0380873.

Rapid exchange catheters are commonly used in Percutaneous Transluminal Coronary Angioplasty (PTCA) procedures, in which obstructed blood vessels are typically dilated by a distal balloon mounted on the catheter's distal end. A stent is often placed at the vessel's dilation zone to prevent reoccurrences of obstruction therein. The dilation balloon is typically inflated via an inflation lumen which extends longitudinally inside the catheter's shaft between the dilation balloon and the catheter's proximal end.

The guide-wire lumen passes within a smaller section of the catheter's shaft length and it is accessed via a lateral port situated on the catheter's shaft. This arrangement, wherein the guide-wire tube is affixed to the catheter's shaft at the location of its lateral port, usually prevents designers from developing new rapid exchange catheter implementations which requires manipulating its inner shaft. For example, extending or shortening the catheter's length during procedures may be advantageously exploited by physicians to distally extend the length of the catheter into a new site after or during its placement in the patient's artery, for example in order to assist with the passage of tortuous vessels or small diameter stenoses, or to allow in-situ manipulation of an inflated balloon at the distal end of the catheter.

Published international patent application, publication No. WO 2005/102184 discloses a catheter having a rollable expandable element. Published international patent applications, publication Nos. WO 2007/004221, WO 2007/042935, WO 2008/004238 and WO 2008/004239, all five published international applications are incorporated herein by reference in their entirety for all purposes, disclose various types of catheters and catheter systems having intussuscepting balloon-like inflatable members which may be used, inter alia, to treat plaque by balloon inflation while efficiently and safely collecting plaque debris and other particulate matter from the lumen of pathologically-involved blood vessels and to remove such particles and particulate matter from the blood vessel.

A problem frequently encountered in the use of inflatable balloons to treat atheromatous plaque in blood vessels is that inflation of the balloon against the wall of the blood vessel may sometimes cause some damage to the blood vessel wall in the region of contact between the balloon and the blood vessel walls. Physicians are therefore usually reluctant to use balloons longer that the length necessary for treating most of the plaque. However, when the intussuscepting balloons as disclosed, inter alia, in WO 2005/102184 and WO 2007/004221 are used for treating plaque (by expanding the balloon placed inside the plaque region or by other methods) and for trapping and internalizing debris particles or secretions and fluids from inside a treated blood vessel, one would like to increase the capacity of the balloon to trap and include debris particles in it's intussuscepted (invaginated) state without increasing the surface of the balloon which will be in contact with the non-pathologically involved portions of the walls of the blood vessel flanking the treated area.

International patent applications PCT/IL2009/000667 and PCT/IL2009/000668 commonly assigned to the assignee of the present application and incorporated herein by reference in their entirety for all purposes, disclose various types of over the wire (OVT) catheters having stepped and corrugated types of intussuscepting balloons, respectively.

Another concern which may be encountered in the use of intussuscepting balloons, such as, for example, the balloons disclosed in WO 2005/102184 and WO 2007/004221 is that in balloons in which it is desired to have the distal part of the balloon intussuscept, it is important to ensure that the distal end of the balloon (the end attached to the inner tube of the catheter) collapses preferentially at a lower pulling force than the force required to collapse the proximal end of the balloon (the end of the balloon attached to the outer tube of the catheter) to ensure proper intussuscepting of the distal end of the balloon. (The proximal and distal ends of the balloon are defined as described in WO 2005/102184 and WO 2007/004221). However, in other balloons in which it is desired to have the proximal part of the balloon intussuscept, it is important to ensure that the proximal end of the balloon collapses preferentially at a lower pulling force than the force required to collapse the distal end of the balloon to ensure proper intussuscepting of the proximal end of the balloon.

Published International Patent Application, Publication No. WO 2005/102184 discloses a catheter having a rollable expandable element. Published International Patent applications, Publication Nos. WO 2007/004221, WO 2007/042935, WO 2008/004238 and WO 2008/004239, disclose various types of catheters and catheter systems having intussuscepting balloon-like inflatable members which may be used, inter alia, to treat plaque by balloon inflation while efficiently collecting plaque debris and other particulate matter from the lumen of pathologically-involved blood vessels or other different body internal passages and to remove such particles and particulate matter from the blood vessel or body passage.

Such inflatable intussuscepting balloons may be used to treat plaque by inflating and expanding the balloons after their placement in the plaque region of a blood vessel. Typically, the maximal outer diameter of the balloon in the fully inflated state is limited by the transversal size (or diameter) of the treated blood vessel. Therefore, if one desires to increase the volume available in the intussuscepted balloon for including debris particles and plaque particulates within the space formed, one needs to increase the length of the balloon. However, the length of an inflatable balloon having a uniform cross-sectional area will disadvantageously also increase the length of the balloon surface in contact with the blood vessel walls during the fully inflated state of the balloon. Moreover, from the clinical point of view, it is desirable to minimize the length of the balloon portion which would be placed in direct contact with the surface of the blood vessel during the plaque treatment phase (in which the balloon is expanded), as one would like to minimize the possible damage to the blood vessel wall which may be caused by the expansion of the balloon and its contact with the plaque and the associated blood vessel wall.

Thus, there is a need to increase the total volume within the internal space (cavity) formed within the balloon in its intussuscepted (invaginated) state, without overly increasing the area of contact of the fully inflated balloon with the walls of the blood vessel during plaque treatment.

### SUMMARY OF THE INVENTION

There is therefore provided, in accordance with an embodiment of the catheter of the present application, a rapid exchange balloon catheter. The catheter includes an outer conduit and an inner conduit disposed within the outer conduit and suitable for total or partial passage over a guide-wire. The inner conduit includes at least one movable part movably disposed within the lumen of the outer conduit. The inner conduit includes a proximal angled portion piercing the wall of the outer conduit and a distal end extending beyond the distal end of the outer conduit. The catheter also includes an inflatable balloon having a proximal margin sealingly attached to the outer surface of the distal end of the outer conduit and a distal margin sealingly attached to the outer surface of the portion of the inner conduit that extends beyond the distal end of the outer conduit. The inflatable balloon includes a first portion having a first diameter and at least a second portion having a second diameter smaller than the first diameter. The catheter also includes means for axially moving the at least one movable part of the inner conduit within the outer conduit, means for the introduction of an expansion fluid into the space formed between the outer conduit and the inner conduit and therefrom into the lumen of the balloon and for the removal of the fluid from the space and from the lumen and means for permitting unhindered axial movement of the at least one movable part of the inner conduit within the outer conduit, such that the movement is not hindered by the passage of the angled portion of the inner conduit through the outer conduit.

Furthermore, in accordance with another embodiment of the catheter, the means for axially moving include one or more elongated moving members, the distal end(s) thereof being attached to the at least one movable part of the inner conduit, and the proximal end(s) thereof extending beyond the proximal end of the outer conduit.

Furthermore, in accordance with another embodiment of the catheter, the distal portion of the balloon is capable of intussuscepting upon proximal movement of the at least one movable part of the inner conduit in relation to the outer conduit.

Furthermore, in accordance with another embodiment of the catheter, the catheter also includes means for reducing pressure changes within the space upon axial movement of the at least one movable part of the inner conduit in relation to the outer conduit.

Furthermore, in accordance with another embodiment of the catheter, the means for reducing pressure changes include a piston-like member slidably disposed within the proximal end of the outer conduit. The piston-like member is connected to the means for axially moving, such that upon operation of the means for axially moving the piston-like member is caused to move either distally or proximally, changing the volume of the outer conduit.

Furthermore, in accordance with another embodiment of the catheter, the means for permitting unhindered axial movement include a sealing sleeve sealingly attached to the angled portion of the inner conduit and slidably fitted around the outer conduit, such that the angled portion of the inner conduit passes firstly through an elongated aperture in the wall of the outer conduit, and secondly through a tightly sealed aperture in the sealing sleeve, so that upon axial movement of the at least one movable part of the inner conduit, the sealing sleeve is capable of preventing leaking of inflation fluid through the elongated aperture.

Furthermore, in accordance with another embodiment of the catheter, the means for permitting unhindered axial movement of the inner conduit is provided by a two-part inner conduit construction, wherein a first proximal part of the two-part inner conduit includes a non-movable inner tube including the angled portion, and a second distal part of the two-part inner conduit includes a slidable internal tube disposed within the non-movable inner tube.

Furthermore, in accordance with another embodiment of the catheter, the means for permitting unhindered axial movement of the inner conduit is provided by a two-part inner conduit construction, wherein a first proximal part of the two-part inner conduit includes a non-movable inner tube including the angled portion, and a second distal part of the two-part inner conduit includes a slidable internal tube disposed over the non-movable inner tube.

Furthermore, in accordance with another embodiment of the catheter, the means for permitting unhindered axial movement of the inner conduit is provided by a two-part inner conduit construction, wherein the two-part inner conduit includes a non-movable inner tube including the angled portion and a slidable intermediate tube movably disposed between the non movable inner tube and the outer conduit. The intermediate tube has an elongated longitudinal opening on its side through which the angled portion passes. The distal end of the intermediate tube is the portion of the inner conduit that extends beyond the distal end of the outer conduit. The distal margin of the balloon is sealingly attached to the outer surface of the distal end of the intermediate tube and the proximal end of the intermediate tube sealingly passes through and extends beyond the proximal end of the outer conduit such that the means for axially moving includes the proximal end of the intermediate tube.

Furthermore, in accordance with another embodiment of the catheter, the means for permitting unhindered axial movement of the inner conduit is provided by a three-part inner conduit construction. The three-part inner conduit includes, a first non-movable hollow tube including the angled portion at its proximal portion and having a distal end, a second non-movable hollow inner tube having a proximal end and a distal end, the second inner tube is sealingly disposed within the distal end of the first non-movable inner tube, and a third slidable inner tube slidably disposed over the distal end of the second non-movable hollow tube, the third slidable inner tube has a distal end extending beyond the distal end of the outer conduit. The distal margin of the balloon is attached to the outer surface of the portion of the distal end of the third inner tube extending beyond the distal end of the outer conduit.

Furthermore, in accordance with another embodiment of the catheter, the outer conduit includes a lateral opening therein. The means for permitting unhindered axial movement includes a sealing sleeve internally disposed within the outer conduit and attached to the angled portion of the inner conduit. The sealing sleeve is sealingly fitted within the outer conduit such that the angled portion of the inner conduit passes firstly through the wall of the sealing sleeve, and secondly through the lateral opening of the outer conduit, so that upon axial movement of the inner conduit and the sealing sleeve, the sealing sleeve is capable of preventing leaking of inflation fluid through the lateral opening.

Furthermore, in accordance with another embodiment of the catheter, the inflatable balloon is characterized by having, in its inflated state, a shape which is capable of guiding the intussuscepting of the distal end thereof upon proximal movement of the at least one movable part of the inner conduit in relation to the outer conduit.

Furthermore, in accordance with another embodiment of the catheter, the first portion includes at least a first cylindrical portion. The second portion is proximal to the first portion and includes at least a second cylindrical portion having in the inflated state a diameter smaller than the diameter of the first cylindrical portion in the inflated state.

Furthermore, in accordance with another embodiment of the catheter, the second portion also includes at least two frusto-conical portions flanking the distal and the proximal sides of the second cylindrical portion.

Furthermore, in accordance with another embodiment of the catheter, the second portion includes at least one frusto-conical portion.

Furthermore, in accordance with another embodiment of the catheter, the second portion includes one or more portions selected from, cylindrical portions, frusto-conical portions, concave tapering portions, convex tapering portions, and combinations thereof.

Furthermore, in accordance with another embodiment of the catheter, the length of the second proximal portion is equal to or larger than the length of the first portion.

Furthermore, in accordance with another embodiment of the catheter, the at least a second portion includes a second portion proximal to the first portion and a third portion distal to the first portion and the inflated diameter of the third portion is smaller than the diameter of the first portion.

Furthermore, in accordance with another embodiment of the catheter, the length of the second proximal portion is equal to or larger than the combined length of the first portion and the third portion.

Furthermore, in accordance with another embodiment of the catheter, at least part of the inflatable balloon is corrugated.

Furthermore, in accordance with another embodiment of the catheter, the balloon has a non-uniform wall thickness along its longitudinal axis.

Furthermore, in accordance with another embodiment of the catheter, the at least a second portion is proximal to the first portion of the balloon and the wall thickness of at least part of the first portion is smaller than the wall thickness of at least part of the second portion of the balloon.

Furthermore, in accordance with another embodiment of the catheter, the first portion of the inflatable balloon includes one or more portions selected from dome-like portions, truncated dome-like portions, conical portions, frusto-conical portions, corrugated dome-like portions, corrugated conical portions, corrugated frusto-conical portions, corrugated truncated dome-like portions and combinations thereof.

Furthermore, in accordance with another embodiment of the catheter, the balloon catheter also includes a pressure adjusting mechanism for preventing substantial pressure changes within the space within the catheter and the lumen of the balloon upon proximal axial movement of the at least one movable part of the inner conduit in relation to the outer conduit.

Furthermore, in accordance with another embodiment of the catheter, the pressure adjusting mechanism is selected from,

a pressure adjusting mechanism including a syringe-like structure disposed at the proximal end of the catheter. The syringe-like structure includes a piston-like member. The syringe-like structure is in fluidic communication with the internal space of the catheter. The piston like member is movably disposed within the syringe-like structure and is mechanically coupled to the means for axially moving the at least one movable part of the inner conduit, such that when the movable part of the inner conduit is moved proximally, the amount of inflation fluid ejected from the balloon during the intussuscepting thereof is accommodated within the syringe-like structure,

an outlet in fluidic communication with the lumen of the inflatable balloon and having an opening and a compliant member sealingly attached to the opening for at least partially relieving over-pressure in the lumen,

an over-pressure valve outlet in fluidic communication with the lumen of the inflatable balloon and an over-pressure valve disposed within the over-pressure outlet to allow discharging of fluid from the lumen when over-pressure conditions develop in the lumen,

an expandable or inflatable portion of the outer conduit, capable of expanding when over-pressure conditions occur in the lumen of the balloon to at least partially relieve the over-pressure in the lumen, and

a hydraulic accumulator configured for being controllably fluidically connected and disconnected from the space and the lumen of the balloon.

Furthermore, in accordance with another embodiment of the catheter, the first portion of the balloon is distal to the at least a second portion and is characterized by having, in its inflated state, a distal end shape selected from the group consisting of a distal taper with a rounded distal extremity, a dome-like portion, a truncated dome-like portion, a conical portion, a frusto-conical portion, a corrugated dome-like portion, a corrugated conical portion, a corrugated frusto-conical portion, and a corrugated truncated dome-like portion.

There is also provided, in accordance with an embodiment of the methods of the present application, a method of constructing an intussusceptible balloon rapid exchange catheter. the method includes the steps of,

providing a catheter having an outer conduit and an inner conduit disposed within the outer conduit and suitable for total or partial passage over a guide-wire. The inner conduit includes at least one movable part movably disposed within the lumen of the outer conduit. The inner conduit includes a proximal angled portion piercing the wall of the outer conduit. A distal end of the inner conduit extends beyond the distal end of the outer conduit,

providing an inflatable balloon having a proximal margin and a distal margin. The inflatable balloon includes a first portion having a first diameter and at least a second portion having a second diameter smaller than the first diameter, and

sealingly attaching the proximal margin of the balloon to the outer surface of the distal end of the outer conduit and sealingly attaching the distal margin of the balloon to the outer surface of the portion of the inner conduit that extends beyond the distal end of the outer conduit, such that the lumen of the balloon is in fluidic communication with the space defined between the outer conduit and the inner conduit, The attaching is performed such that the distal end of the balloon is capable of intussuscepting upon proximal movement of at least one movable part of the inner conduit in relation to the outer conduit.

There is also provided, in accordance with an embodiment of the methods of the present application, a method for collecting debris from an internal passage of a mammalian subject. The method includes the steps of:

a) Inserting a rapid exchange balloon catheter into the internal passage, and advancing the catheter until the distal end thereof has reached a site at which it is desired to collect debris. The rapid exchange balloon catheter includes an outer conduit, and an inner conduit disposed within the outer conduit and suitable for total or partial passage over a guide-wire. The inner conduit includes at least one movable part movably disposed within the lumen of the outer conduit. The inner conduit includes a proximal angled portion piercing the wall of the outer conduit and a distal end extending beyond the distal end of the outer conduit. The catheter also includes an inflatable balloon having a proximal margin sealingly attached to the outer surface of the distal end of the outer conduit, and a distal margin sealingly attached to the outer surface of the portion of the inner conduit that extends beyond the distal end of the outer conduit. The inflatable balloon includes a first portion having a first diameter and at least a second portion having a second diameter smaller than the first diameter. The catheter also includes means for axially moving the at least one movable part of the inner conduit within the outer conduit, means for the introduction of an expansion fluid into the space formed between the outer conduit and the inner conduit and therefrom into the lumen of the balloon and for the removal of the fluid from the space and from the lumen, and means for permitting unhindered axial movement of the at least one movable part of the inner conduit within the outer conduit, such that the movement is not hindered by the passage of the angled portion of the inner conduit through the outer conduit.

b) Inflating the balloon with expansion fluid.

c) Moving the at least one movable part of the inner conduit in a proximal direction, such that the distal end of the first portion of the balloon collapses and the balloon intussuscepts forming a cavity for collecting the debris.

d) Deflating the balloon to increase the volume of the cavity to collect and trap additional debris within the cavity, and

e) Removing the balloon catheter from the internal passage together with the entrapped debris.

Furthermore, in accordance with an embodiment of the method, the internal passage is a blood vessel.

There is also provided, in accordance with an embodiment of the methods of the present application, a method for collecting debris resulting from treatment of a diseased target region of an internal passage of a mammalian subject, The method includes the steps of:

a) Inserting a rapid exchange balloon catheter into the internal passage. The rapid exchange balloon catheter includes an outer conduit. The catheter also includes an inner conduit disposed within the outer conduit and suitable for total or partial passage over a guide-wire. The inner conduit includes at least one movable part movably disposed within the lumen of the outer conduit. The inner conduit includes an angled portion piercing the wall of the outer conduit and the distal end of the inner conduit extends beyond the distal end of the outer conduit. The catheter also includes an inflatable balloon having a proximal margin sealingly attached to the outer surface of the distal end of the outer conduit and a distal margin sealingly attached to the outer surface of the portion of the inner conduit that extends beyond the distal end of the outer conduit. The inflatable balloon includes a first portion having a first diameter and at least a second portion having a second diameter smaller than the first diameter. The catheter also includes means for axially moving the at least one movable part of the inner conduit within the outer conduit. The catheter also includes means for the introduction of an expansion fluid into the space formed between the inner surface of the outer conduit and the outer surface of the inner conduit and therefrom into the lumen of the balloon and for the removal of the fluid from the space. The catheter also includes means for permitting unhindered axial movement of the at least one movable part of the inner conduit within the outer conduit, such that the movement is not hindered by the passage of the angled portion of the inner conduit through the outer conduit, and advancing the catheter to position the balloon within at least part of the diseased target region.

b) Inflating the balloon with expansion fluid to treat at least part of the diseased target region contacted by the first portion, such that at least some of the debris formed during the inflating is attached to the outer surface of the first portion.

c) Proximally moving the at least one movable part of the inner conduit of the balloon catheter, such that the distal end of the balloon collapses and the balloon intussuscepts, trapping at least some of the debris within a cavity formed therein. At least part of the outer surface of the first portion is internalized within the cavity.

d) Deflating the balloon to increase the volume of the cavity and to trap additional debris therewithin.

e) Removing the balloon catheter from the internal passage, together with the entrapped debris.

Furthermore, in accordance with an embodiment of the method, the internal passage is a blood vessel and the diseased portion includes an atheromatous plaque.

Furthermore, in accordance with an embodiment of the method, the at least second portion of the balloon is longer than the first portion of the balloon, and the entire outer surface of the first portion is internalized within the cavity in the step of proximally moving.

There is also provided, in accordance with an embodiment of the methods of the present application, a method for collecting debris from an internal passage of a mammalian subject. The method includes the steps of,

inserting an inflatable balloon catheter portion into the internal passage, and advancing the catheter until the distal end thereof has reached a site at which it is desired to collect debris,

inflating the inflatable balloon with expansion fluid;

moving at least one movable part of an inner conduit of the catheter in a proximal direction for collapsing the distal end of the inflatable balloon to form an intussuscepted balloon having a cavity therein into which cavity the debris is collected and entrapped,

deflating the intussuscepted balloon; and

removing the balloon catheter from the internal passage, together with the entrapped debris.

Furthermore, in accordance with an embodiment of the method, the internal passage is a blood vessel.

Furthermore, in accordance with an embodiment of the method, the step of moving includes moving the at least one movable part of the inner conduit in a proximal direction to form the cavity, such that all the outer surface of the first portion of the balloon is disposed within the cavity to increase retention of the debris.

Furthermore, in accordance with an embodiment of the method, the catheter includes a mechanism for reducing pressure changes within the catheter when the movable part of the inner conduit is moved proximally within the outer conduit while the balloon is inflated and the fluid port is closed, and the step of moving includes moving the movable part of the inner conduit in a proximal direction, for collapsing the distal end of the balloon to form a cavity within the balloon into which debris is collected and entrapped without inducing substantial pressure changes within the lumen of the balloon during the intussuscepting of the balloon.

Furthermore, in accordance with an embodiment of the method, the internal passage is an occluded blood vessel and the step of inflating includes inflating the balloon while the balloon is disposed near or within an atheromatous plaque of the occluded blood vessel. The inflating is performed such that at least part of the first portion of the balloon is pushed against the plaque and at least some debris from the plaque adheres to the outer surface of the first portion and is internalized within the cavity formed in the step of pulling.

There is also provided, in accordance with an embodiment of the methods of the present application, a method for treating a diseased target region of an internal passage of a mammalian subject and for collecting debris from the internal passage. The method includes the steps of:

inserting a balloon catheter into the internal passage. The catheter includes an inflatable intussusceptable balloon. The balloon includes at least a first portion having a first diameter and at least a second portion having a second diameter smaller than the first diameter, and advancing the catheter until the distal end thereof reaches the target region.

inflating the balloon such that the first portion contacts the target region to treat the target region while the at least second portion does not contact the walls of the internal passage,
collapsing the distal end of the intussusceptable balloon while the balloon is in an inflated state to form a cavity within the balloon into which debris is collected and entrapped,

deflating the intussuscepted balloon to increase the volume of the internal cavity, and
removing the balloon catheter from the internal passage together with debris entrapped within the cavity.

Furthermore, in accordance with an embodiment of the method, the step of deflating includes deflating the intussuscepted balloon to increase the volume of the internal cavity and to further trap additional debris in the increased volume of the internal cavity.

Furthermore, in accordance with an embodiment of the method, the step of deflating includes deflating the intussuscepted balloon to increase the volume of the internal cavity and to create suction assisting the collecting of additional debris into the cavity.

Furthermore, in accordance with an embodiment of the method, the step of collapsing includes intussuscepting the balloon such that at least some of the external surface of the first portion in internalized within the cavity and at least some debris attached to the external surface is trapped within the cavity.

Furthermore, in accordance with an embodiment of the method, the catheter includes a mechanism for reducing pressure changes within the catheter and the step of collapsing includes collapsing the distal end of the intussusceptable balloon while the balloon is in an inflated state to form a cavity within the balloon into which debris is collected and entrapped, without causing a substantial pressure change within the balloon during the collapsing.

Furthermore, in accordance with an embodiment of the method, the at least second portion includes a proximal second portion of the balloon. The length of the second portion is greater than the length of the first portion, and the step of collapsing includes collapsing the distal end of the intussusceptable balloon while the balloon is in an inflated state to form the cavity until the entire external surface of the first portion is completely internalized within the cavity.

Furthermore, in accordance with an embodiment of the method, the at least a second portion includes a proximal second portion of the balloon. The length of the first portion is greater than the length of the first portion, and the step of collapsing includes collapsing the distal end of the intussusceptable balloon while the balloon is in an inflated state to form the cavity until at least part of the external surface of the first portion is internalized within the cavity.

Furthermore, in accordance with an embodiment of the method, the internal passage is a blood vessel, the target region is an occluded region of the blood vessel and at least some of the debris is formed during an opening of the occluded region by the first portion of the balloon.

There is also provided, in accordance with an embodiment of the catheter of the present application, a rapid exchange balloon catheter. The catheter includes an outer conduit and an inner conduit disposed within the outer conduit and suitable for total or partial passage over a guide-wire. The inner conduit includes at least one movable part movably disposed within the lumen of the outer conduit. The inner conduit has a proximal angled portion piercing the wall of the outer conduit and a distal end extending beyond the distal end of the outer conduit. The catheter also includes an inflatable balloon having a proximal margin sealingly attached to the outer surface of the distal end of the outer conduit, and a distal margin sealingly attached to the outer surface of the portion of the inner conduit that extends beyond the distal end of the outer conduit. The inflatable balloon includes a first portion having a first diameter and at least a second portion having a second diameter smaller than the first diameter. The catheter also includes a moving mechanism for axially moving the at least one movable part of the inner conduit within the outer conduit. The moving mechanism permits unhindered axial movement of the at least one movable part of the inner conduit within the outer conduit, such that the movement is not hindered by the passage of the angled portion of the inner conduit through the outer conduit, The catheter also includes a fluid port for the introduction of an expansion fluid into the space formed between the outer conduit and the inner conduit and therefrom into the lumen of the balloon and for the removal of the fluid from the space and from the lumen.

Finally, in accordance with an embodiment of the catheter of the present application, the catheter also includes a pressure regulating mechanism for reducing pressure changes within the space of the catheter and within the balloon upon axial movement of the at least one movable part of the inner conduit in relation to said outer conduit.

### BRIEF DESCRIPTION OF THE DRAWINGS

The invention is herein described, by way of example only, with reference to the accompanying drawings, in which like components are designated by like reference numerals, wherein:

FIGS. 1A-1D are schematic cross sectional views illustrating a rapid exchange catheter having a stepped balloon in which the distal section of the inner tube includes an internal slidable tube according to an embodiment of the catheters of the present application;

FIG. 1E is a cross sectional view illustrating a part of an additional embodiment of a rapid exchange catheter having a piston-like member for preventing pressure changes within the catheter during retraction of a slidable tube of the catheter;

FIGS. 1F-1G are schematic cross-sectional diagrams illustrating parts of a rapid exchange catheter system including an inflatable stepped intussusceptible balloon and a pressure regulating mechanism in accordance with other embodiments of the catheter systems of the present application;

FIGS. 2A-2C are schematic cross sectional views illustrating a rapid exchange catheter having a stepped balloon in which the diameter of the distal portion of a fixed inner tube is adapted to receive an internal slidable tube, in accordance with another embodiment of the rapid exchange catheter of the present application;

FIG. 3 is a schematic cross sectional view illustrating a rapid exchange catheter with a stepped balloon, in which an external slidable tube slides over the distal portion of a fixed inner tube, in accordance with yet another embodiment of the catheter of the present application;

FIG. 4 is a schematic cross sectional view illustrating a rapid exchange catheter with a stepped balloon, in which the diameter of the distal portion of a fixed part of an inner tube is adapted to be received within an external slidable tube according to yet another embodiment of the catheter of the present application;

FIG. 5 is a schematic cross sectional view illustrating a rapid exchange catheter with a stepped balloon in which an inner tube includes a fixed two part proximal tube and an external slidable tube sliding over the distal part of the fixed two part proximal tube, according to yet another embodiment of the catheter of the present application;

FIGS. 6A-6C are schematic cross sectional views illustrating a rapid exchange catheter with a stepped balloon and a fixed inner tube which is encompassed by a slidable intermediate tube, according to yet another embodiment of the catheter of the present application;

FIGS. 7A-7B are schematic cross sectional views illustrating a rapid exchange catheter with a stepped balloon and a movable inner tube attached to an external slidable sealing sleeve, in accordance with yet another embodiment of the catheter of the present application;

FIG. 7C is a schematic cross sectional view illustrating part of a rapid exchange catheter with a stepped balloon and a movable inner tube attached to an internal slidable sealing sleeve, in accordance with still another embodiment of the catheter of the present application;

FIG. 8 is a schematic side view of a tapered stepped sleeve-like element usable in a balloon catheter having an expandable intussusceptible stepped balloon, in accordance with one embodiment of the balloon catheters of the present application;

FIGS. 9-12 are schematic side views and cross-sectional views, illustrating various different possible embodiments of sleeve-like elements usable as inflatable balloons in the catheters and catheter systems of the present application;

FIGS. 13-14 are schematic cross-sectional diagrams illustrating two different embodiments of corrugated stepped tapering sleeve-like elements suitable for constructing balloon catheters having a corrugated stepped tapering intussusceptible balloons in accordance with additional embodiments of the sleeve-like elements and balloon catheters of the present application;

FIG. 15 is a schematic cross-sectional diagram illustrating a corrugated stepped tapering sleeve-like element having a non-uniform wall thickness usable in constructing catheters having a tapering intussusceptible balloon, in accordance with an embodiment of the balloon catheters of the present application;

FIGS. 16-20 are schematic cross-sectional diagrams illustrating a catheter including an intussusceptible balloon having a stepped tapering structure and several different steps of a method for using the catheter for treating atheromatous plaque in a blood vessel and for removing fluid and/or debris particles out of the treated blood vessel, in accordance with an embodiment of the catheter and method of use thereof of the present application;

FIG. 21 is a schematic cross sectional diagram illustrating a step of a method of use of the catheter system of the present application for anchoring the catheter against the walls of a blood vessel prior to the insertion of a plaque treating device through a lumen within the catheter; and

FIG. 22 is a schematic flow chart illustrating the steps of a method for using any of the catheters with stepped intussuscepting balloons of the present application for (optionally) treating a body passage and for removal of debris and/or particulate matter from a body passage, in accordance with an embodiment of the methods of the present application.

### DETAILED DESCRIPTION OF THE INVENTION

Notation Used Throughout

The following notation is used throughout this document.

| Term | Definition |
|---|---|
| CA | Cyanoacrylate |
| DCA | Directional coronary atherectomy |
| ELCA | Excimer Laser Coronary Angioplasty |
| mm | millimeter |
| OVT | Over the wire |
| PA | Polyamide |
| PE | Polyethylene |
| PEBA | Polyether block amides |
| PET | Polyethylene terephthalate |
| PLOSA | Physiologic low stress angioplasty |
| RE | Rapid exchange |

The present application discloses catheters and systems including controllably inflatable and controllably intussusceptible balloons having non-uniform cross-sectional areas along their longitudinal dimension. Such balloons may include a middle cylindrical balloon portion having a first diameter designed for contacting the walls of a body passage (such as, but not limited to, a blood vessel), and one or more non-contacting side portions extending longitudinally on one or more sides of the middle portion. The one or more non-contacting portions are designed and implemented such that when the entire balloon is fully inflated, the maximal transversal dimensions of the side portions are smaller than the transversal dimension (diameter) of the middle portion of the balloon. The side portion(s) may have cylindrical and/or conical and/or frusto-conical, and/or rounded dome-like and/or tapering shape(s). The side portion(s) may also have a shape which is a combination of one or more of cylindrical, conical, frusto-conical, dome-like and tapering shapes.

The balloons may have a distal portion having a first diameter of a cylindrical part thereof and at least a proximal portion having a cylindrical part with a diameter that is smaller than the first diameter of the distal portion.

It is noted that the terms “sleeve-like element” and “balloon” in the singular and plural forms are interchangeably used in the present application. The term “sleeve-like element” is typically used throughout the application to refer to the element or balloon before it is assembled into the balloon catheter, while the term “balloon” is used to refer to the same sleeve-like element after it has been assembled into the balloon catheter. However, for the sake of convenience, these two terms may also be used interchangeably in the application irrespective of whether the balloon is show as part of a catheter or not and irrespective of whether the sleeve-like element is shown alone or as attached to a catheter.

It is also noted that the terms “corrugated balloon” and “concertina-like balloon” (in the single as well as the plural forms) are interchangeably used herein to indicate a balloon or an inflatable element or sleeve-like element having multiple folds or corrugations formed at least in a part or a portion thereof. The folds or corrugations may be symmetrical or non-symmetrical and may be of any desired shape such as but not limited to folds having triangular, or rounded, or curved, or sawtooth like cross-sectional shape or any other suitable cross-sectional shape.

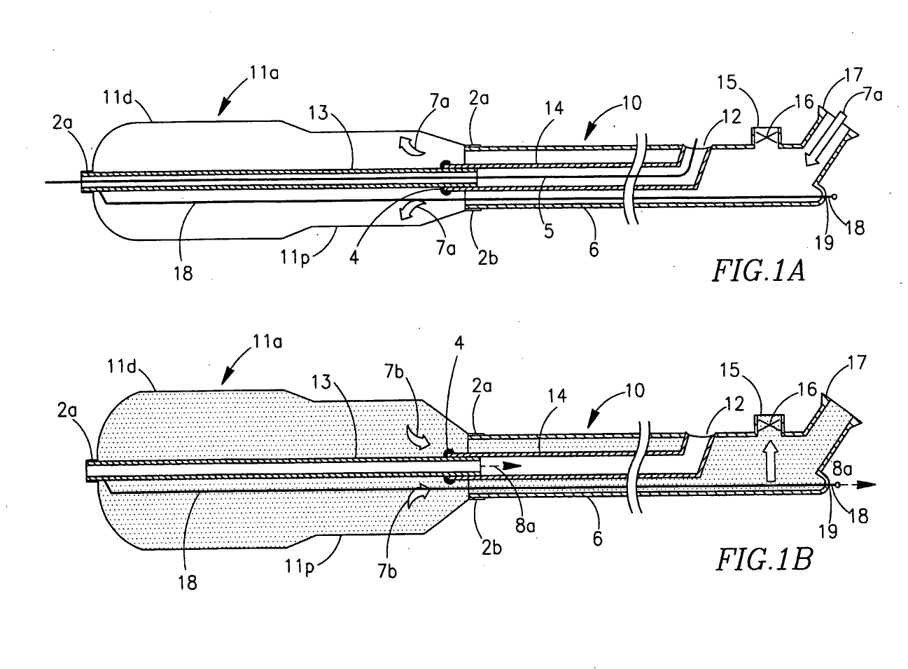

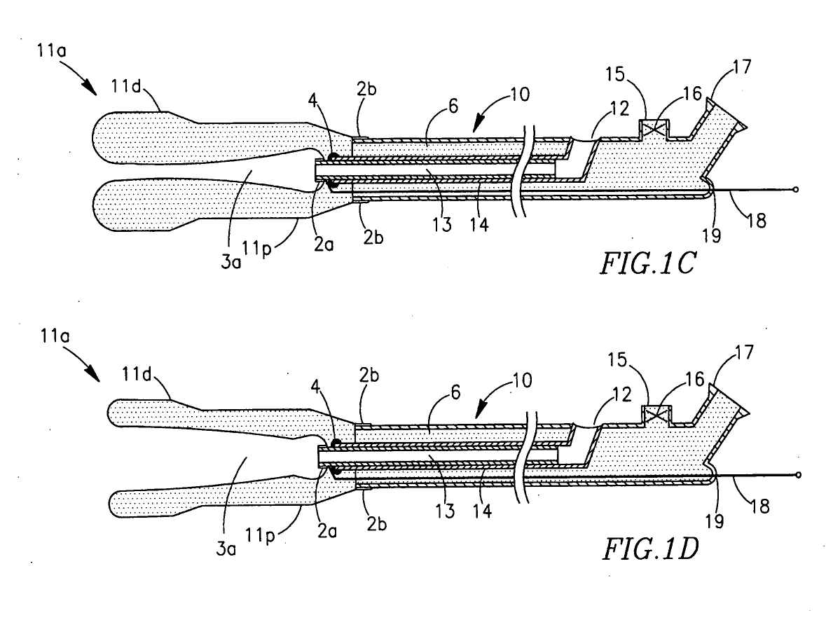

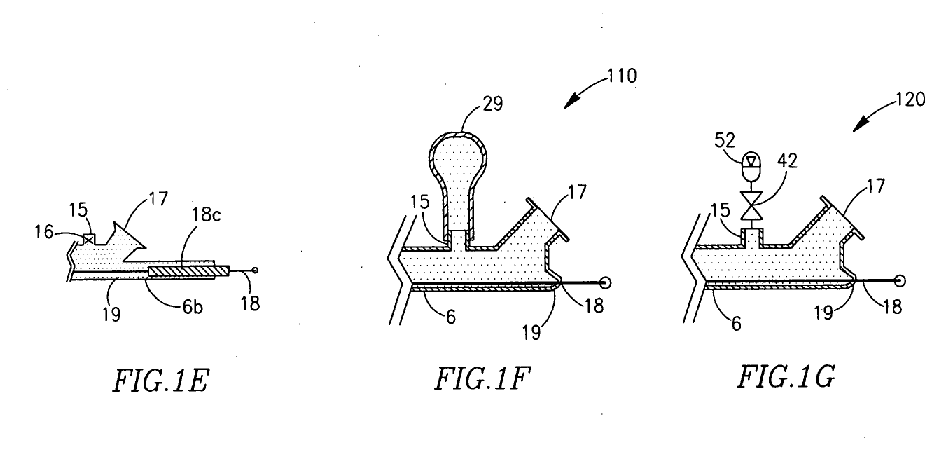

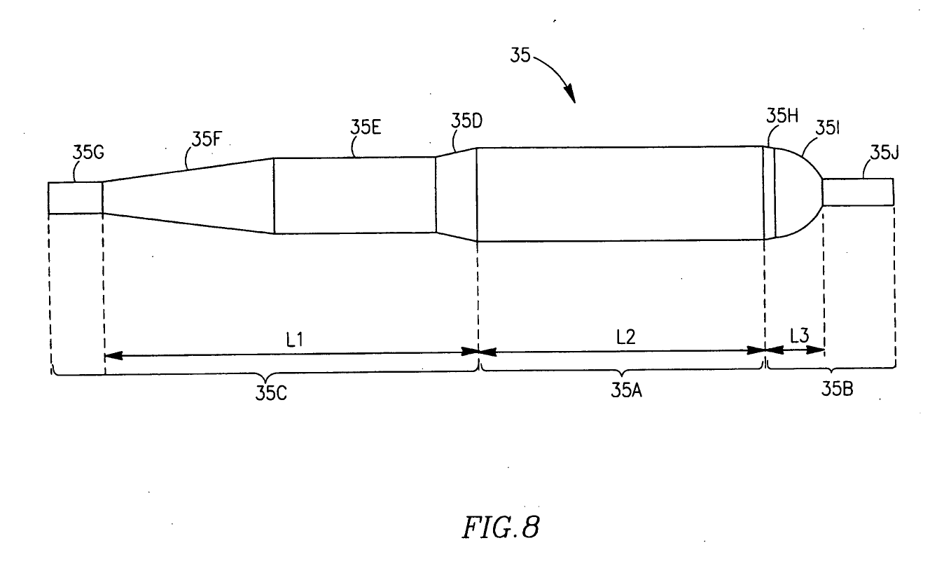

It is also noted that in the following description and in the claims of the present application, the terms “distal” and “proximal” are defined as follows: the catheter side or end which is inserted into the body first is referred to as the distal side or distal end and the trailing side or end of the catheters part of which remains outside the body after insertion of the catheter is referred to as the proximal side. For example, in the balloon catheter 10 of FIG. 1A, the fluid port 17 is disposed on the proximal side of the catheter 10 and the attachment point 2a is disposed near the distal side or distal end of the catheter 10. Similarly, when referring to sides, parts or portions of the balloons (or sleeve-like elements) of the catheters of the present application, the term distal refers to a part, end or portion of the balloon (or sleeve-like element) which is inserted first into the body when the balloon catheter is operated. For example, the corrugated balloon 11a of FIGS. 1A-1D has a distal portion 10d, and a proximal portion 11p. In another example, the balloon 35 of FIG. 8 has a distal portion 35B, a middle portion 35A and a proximal portion 35C. Similarly, the side of the catheter which is first inserted into the body is referred to as the distal catheter side and the side of the catheter which stays out of the body is referred to as the proximal catheter side. This convention is also used for all parts and components of the catheters which longitudinally extend along the catheter axis.

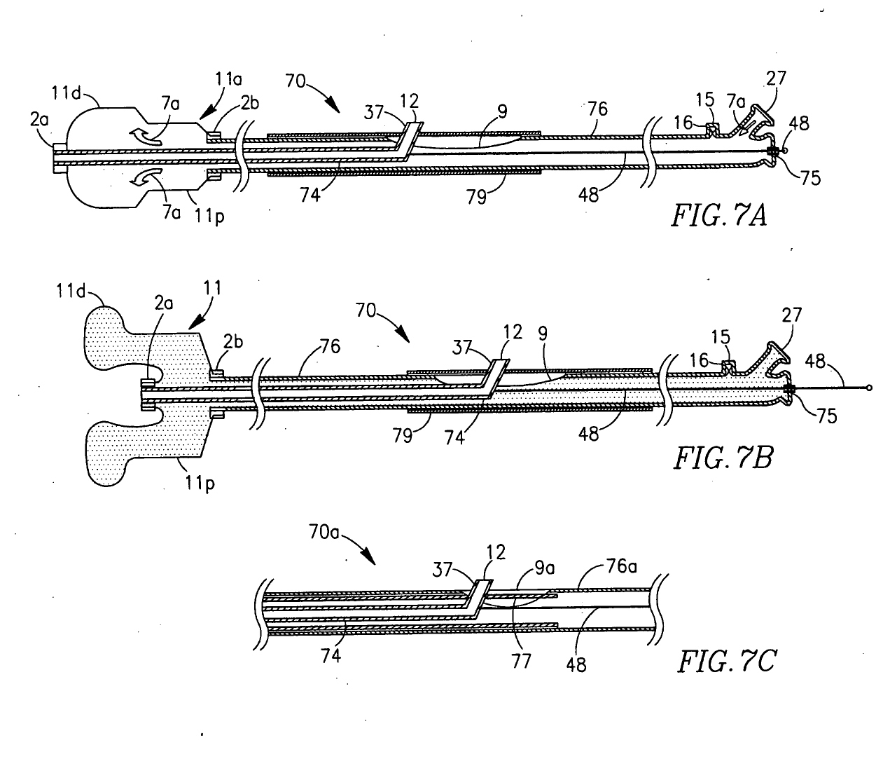

In the following description, the terms “conduit” and “tube” are used interchangeably throughout the application wherein a conduit may include multiple tubes in various relationship thereof. For example, a conduit may include two or three tubes of different diameters wherein one of the tubes may be movably or fixedly disposed within one or more of the other tubes. However, it is noted that the term conduit may also be used to define a single tube (for example, the inner conduits of the catheters 70 and 70a of FIGS. 7A and 7C, respectively, are comprised of a single inner tube 74, as disclosed in detail hereinafter). Thus, as defined herein, the term “conduit” may also refer to a component comprising one or more parts or tubes in different moving or fixed relationships therebetween.

Furthermore, the term “outer conduit” may be used to define the hollow shaft of the catheters of the present application (such as, but not limited to, the hollow shafts 6, 63 and 76 of FIGS. 1A, 6A and 7A, respectively).

The present invention provides rapid exchange catheter implementations in which the length of a distal portion of the catheter and the shape and/or volume of its distal balloon may be manipulated during procedures carried out therewith. The catheters have a stepped balloon which has a first balloon portion with a large diameter and one or more additional portions which diminish in diameter proximally and/or distally of the first balloon portion. These additional portion(s) may extend proximally or distally or proximally and distally of the first larger diameter portion. Preferably, but not obligatorily the proximal balloon portion has one or more cylindrical portions having a diameter smaller than the diameter of the first portion. However, in accordance with other embodiments of the balloons, the proximal portion or the distal portion may have a continuously tapering profile that diminishes in diameter in a direction away from the first portion having the largest diameter.

The stepped balloons of the present application provide have the advantages over the use of intussuscepting balloons of similar dimensions having uniform diameters in that the length of the largest diameter first portion of the balloon that is used for treating the target region (such as, by being expanded and contacting an occluded portion of a blood vessel to achieve compaction of the occlusion) may be optimized (by using a balloon with a minimal length of the first portion so that only the necessary length of the treated occlusion will be contacted by the first portion without shortening the total length of the entire balloon (which therefore contributes to the overall length and internal volume of the cavity formed by intussuscepting of the balloon. This reduces possible damage which may be caused to the walls of the treated blood vessel by reducing the length of the portion of the balloon which contacts the blood vessel wall following balloon inflation while still allowing the maintaining of a sufficient length of the other (narrower) proximal portions of the balloon, so that a long cavity may, be achieved during intussuscepting of the balloon increasing the cavity volume available for internalizing and capturing potentially harmful debris.

In general, the rapid exchange catheter of the invention comprises an outer catheter shaft and an inner conduit provided therein. The inner conduit has a fixed inner tube wherein the lumen of the inner tube may be accessed via a lateral port provided on the catheter's shaft. The inner conduit may also include at least one movable part which may be slidably disposed over or within the distal part of the fixed part of the inner conduit. In some of the embodiments of the catheter described herein the inner tube of the inner conduit is affixed to the catheter's outer shaft and the catheter's length and its balloon are manipulated by a unique construction of the inner conduit. In these constructions the catheter's inner conduit may comprise a slidable distal tube that may be moved by the operator, distally or proximally relative to the catheter's outer shaft, by a moving mechanism, such as, for example, an elongated moving member attached to the movable part of the inner conduit. Alternatively, the inner tube may be disposed within the lumen of a slidable intermediate tube which may be moved by the operator distally or proximally relative to the catheter's shaft.

In further embodiments of the invention a unique catheter construction is disclosed that provides a movable inner tube affixed to a slidable sealing sleeve which allows the operator to move the inner tube distally or proximally relative to the catheter's outer shaft and thereby manipulate its length and balloon.

FIGS. 1A-1D are schematic cross sectional views illustrating a rapid exchange catheter having a stepped balloon in which the distal section of the inner tube includes an internal slidable tube according to an embodiment of the catheters of the present application.

Turning to FIG. 1A, the catheter 10 includes a hollow outer shaft 6 having an inner tube 14 installed therein, and a slidable internal tube 13 disposed within the inner tube 14 such that the slidable internal tube 13 protrudes distally via the distal opening of the inner tube 14. In this construction, the inner lumens of the inner tube 14 and the slidable internal tube 13 are in communication, providing a continuous inner lumen ending at the distal opening of the slidable internal tube 13. The catheter 10 includes a stepped balloon 11a. The Proximal end of the balloon 11a is attached to the hollow outer shaft 6 at proximal attachment region 2b provided around the outer surface of a distal portion of the shaft 6, and the distal end of the balloon 11a is attached to the slidable internal tube 13 at distal attachment region 2a provided around the outer surface of a distal portion of the slidable internal tube 13. The balloon 11a includes a distal portion 11d having the shape of a cylindrical tube terminating in a rounded dome-like end. The balloon 11a also includes a proximal portion 11p shaped like a cylindrical tube terminating in conical proximal portion. The diameter of the distal portion 11d is substantially larger than the diameter of the proximal portion 11p.

The lumen of inner tube 14 may be accessed via a lateral port 12 provided on hollow outer shaft 6, between the distal and proximal ends thereof. A guide-wire 5 (or any other suitable accessories, instruments or devices for diagnosis or treatment, as is known in the art) may be inserted via lateral port 12, advanced along the inner lumens of the inner tube 14 and the slidable internal tube 13, and may exit the inner lumen of the slidable internal tube 13 through a distal opening thereof. The slidable internal tube 13 is adapted to fit into the inner tube 14 and its diameter is preferably smaller than the diameter of the inner tube 14 such that it seals the distal opening of the inner tube 14 while still permitting distal and/or proximal sliding longitudinal movements of the slidable internal tube 13 within the inner tube 14.

The catheter 10 includes an elongated moving member 18. The elongated moving member 18 is implemented as a rod or wire made from stainless steel or any other suitable strong material. The distal end portion of the moving member 18 is attached to the slidable internal tube 13, allowing the operator to move the slidable internal tube 13 distally or proximally relative to the catheter's outer shaft 6 by pushing or pulling the proximal end of moving member 18. Further sealing of the distal opening of the inner tube 14 may be (optionally) achieved by an annular gasket 4 attached to the surface of the distal end of inner tube 14 such that a distal portion of the gasket 4 is pressed against an annular portion of the outer surface of the slidable internal tube 13.

The proximal portion of the hollow shaft 6 includes a fluid port 17 usable for inflating or deflating the corrugated balloon 11a by flowing an inflation fluid into or out of the fluid port 17 (such as, for example, by attaching an indeflator device to the fluid port 17 as is known in the art and using the indeflator device to inject or withdraw fluid through the fluid port, respectively). The catheter 10 also includes an optional over-pressure (discharge) valve 16 installed in a discharge valve outlet 15, and an aperture 19 for sealingly moving the member 18 distally or proximally therethrough. The moving member 18 passes through and is sealingly disposed within the aperture 19, such that it may be moved proximally and distally within the shaft 6.

During a typical procedure catheter 10 is inserted into a body treatment site in which the corrugated balloon 11a may be inflated by an inflation fluid (entry of the inflation fluid is schematically designated by arrows 7a in FIG. 1A) which flows through the inflation fluid port 17 under pressure, for effecting dilatation or other procedures in the treatment site and/or for anchoring the balloon 11a therein. The pressurized fluids pass through the hollow interior of hollow shaft 6 and reach the interior of the balloon 11a via a distal opening of the shaft 6. In its inflated state, shown in FIG. 1B, the hollow interior of shaft 6 and the internal space of balloon 11a are filled with pressurized inflation fluid. The distal opening of the inner tube 14 is sealed by the slidable internal tube 13 and (optionally) by the gasket 4, preventing leakage of pressurized inflation fluid into the inner tube 14. The pressure of the inflation fluid inside the system presses the gasket 4 and improves the sealing provided by gasket 4. On the other hand, when the pressure of the inflation fluid is reduced, the gasket's grip on the outer surface of slidable internal tube 13 is diminished which makes it easier for the slidable internal tube 13 to slide within the gasket 4.

Turning to FIG. 1B, in operation, the catheter 10 may be inserted into the body and advanced to the target region to be treated. For example, the catheter 10 may be inserted into a blood vessel and placed within an atherosclerotic or occluded region to be treated (region not shown). The balloon 11a may then be inserted into the occluded target region and inflated to treat the occlusion as is known in the art. When the balloon 11a is inflated and expanded, the expanded distal portion 11d or part thereof may come into contact with the plaque of the stenosed blood vessel wall and some of the plaque debris which may have formed during the expansion may adhere to the external surface of the distal portion 11d. As the inflated diameter of the cylindrical part of the portion 11d is larger than the diameter of the cylindrical part of the proximal portion 11p, the proximal portion 11p does not contact the walls of the blood vessel, advantageously reducing the possibility of damage to the blood vessel walls.

The requisite procedure is typically carried out in the inflated state of the balloon 11a. In using the catheter 10 for such procedures the operator may manipulate the catheter length and the shape and volume of the balloon 11a by pulling the moving member 18, moving the slidable internal tube 13 proximally further into the inner tube 14, as schematically indicated by arrows 8a. As a result, the distal end of the balloon 11a collapses and folds internally, as illustrated in FIG. 1C, which increases the pressure of the inflation fluid. Whenever the pressure of the inflation fluid inside the hollow interior of hollow outer shaft 6 and in balloon 11a exceeds a predetermined threshold value, a slender passage of the over-pressure valve 16 is expanded to allow portions of inflation fluid to exit via discharge valve outlet 15 reducing the pressure of inflation fluid below the threshold value.

It is noted that the use of the over-pressure valve 16 and the discharge valve outlet 15 constitutes merely one possible, exemplary means of pressure reduction, and that other different pressure regulating mechanisms for preventing a substantial pressure change within the catheter 10 and the balloon 11a may also be used as is disclosed in detail hereinafter.

The hollow outer shaft 6 is preferably made from a polymer based or metallic material, such as stainless steel 316, nitinol, Nylon®, and the like and it may be manufactured utilizing conventional methods, such as extrusion and laser cutting, or any other manufacturing method known in the art. The diameter of the hollow interior of hollow shaft 6 is generally in the range of 1-2 mm (millimeter), preferably about 1.2 mm, and the diameter of the inflation fluid port 17 is generally in the range of 2-6 mm, preferably about 3 mm. The diameter of discharge valve outlet 15 is generally in the range of 2-6 mm, preferably about 3 mm, and the entire length of the hollow shaft 6 is generally in the range of 500-2000 mm, preferably about 1200 mm.

The inner tube 14 is preferably made from a flexible polymer or metallic material, such as PEBAX®, Nylon®, stainless steel, nitinol, or any other suitable material, and may be manufactured utilizing conventional methods, such as extrusion and laser cutting. The to diameter of the inner lumen of the inner tube 14 is generally in the range of 0.3-1 mm, preferably about 0.8 mm, and its entire length is generally in the range of 100-300 mm, preferably about 120 mm. The slidable internal tube 13 is preferably made from a flexible polymer or metallic type of material, such as pebax, nylon, stainless or nitinol, and it may be manufactured utilizing conventional methods, such as PEBAX®, Nylon®, stainless steel, Nitinol® and the like. The diameter of inner lumen of the slidable internal tube 13 is generally in the range of 0.3-1 mm, preferably about 0.5 mm, and its entire length is generally in the range of 30-150 mm, preferably about 70 mm.

However, the typical dimensions of the various parts of the catheter disclosed herein and the materials used for constructing them are given by way of example only, are not obligatory, and may vary substantially depending, inter alia, on the particular medical application of the catheter, the type and size of the treated bodily passage or blood vessel being treated and other engineering, manufacturing and operating considerations.

In view of the axially-directed stretching and buckling forces exerted on the inner and outer tubes during elongation and shortening of the balloon, the tubes need to be constructed such that they are able to withstand axially-directed forces in the range of between 1 and 30 Newton without undergoing deformation. In order to achieve this aim, the conduits may be constructed of a braided material or reinforced material (made by using any suitable reinforcing method known in the art) or of materials having a defined molecular orientation. The approximate maximum forces that the inner and outer tubes need to withstand (for two difference size ranges of balloon) are as follows:

For 2-4 mm balloons, the tubing should withstand up to 500 gram; polymer tubing made of nylon or pebax reinforced during the manufacturing process may be used.
For 4-6 mm (or larger) balloons, the tubing should withstand forces up to 2 kg. In this case it will be necessary to use a braided tube (polymer tube with metal mesh reinforcement) or a tube reinforced by any other suitable tube reinforcing methods known in the art.

Exemplary results for a representative study of the forces generated during balloon folding for a smooth non-corrugated balloon (in an OVT type catheter) are given in detail in WO 2007/042935 and are therefore not be discussed in detail herein.

The balloon 11a is preferably a type of non-compliant or semi-compliant or low-compliant balloon. It may be manufactured utilizing conventional methods known in the balloon catheter industry from a biocompatible polymer type of material such as nylon 12, PET, PEBAX®, PA12 PEBAX®, PEBA, Nylon® 11 (PA11) and the like. The length of the balloon 11a length is generally in the range of 3-350 mm, preferably about 15-50 mm, and its diameter is generally in the range of 2 to 12 mm, preferably about 3 to 5 mm. However, these dimensions are exemplary only, are not meant to be limiting and other different dimensions may be used in making the catheters disclosed herein depending, inter alia, on the particular application, the materials used and other technical and medical considerations.

The proximal and distal ends of the balloon 11a are preferably sealingly attached to the outer surfaces of the hollow shaft 6 and the slidable internal tube 13, at circumferential attachment regions 2b and 2a respectively, preferably by using a low profile type of adhesion such as thermo bonding, UV adhesives or a cyanoacrylate (CA) based adhesive (such as, for example, the cyanoacrylate adhesive manufactured by Locktite Corporation, USA), however, any other attaching method known in the art may be used. Preferably, but not obligatorily, the balloon should have a burst pressure within the range of 6-24 atmospheres.

The materials and design of the corrugated balloons disclosed herein, such as but not limited, the position, number and type of the corrugated portion(s) of the balloon, the shape of the distal taper and the relationship between the distal and the proximal taper, may assist the balloon to fold smoothly and with relatively low pulling forces. Balloon configurations and designs for smooth non-corrugated balloons where disclosed in details in WO 2007/042935 and are therefore not disclosed in details herein, such configurations and designs may also be used in the corrugated balloons of the present application where relevant.

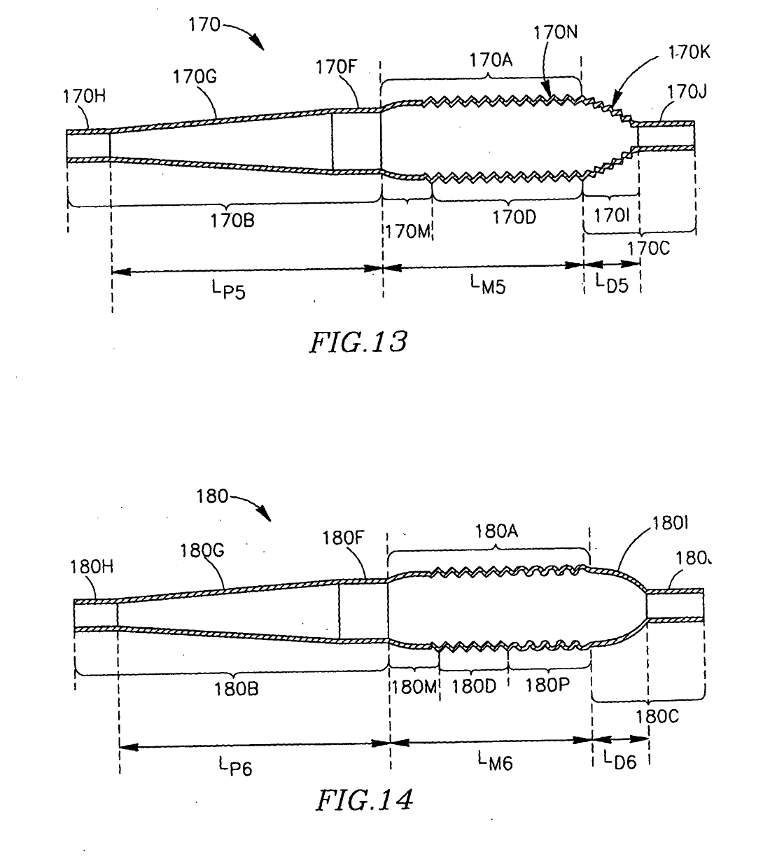

For example, a tapered balloon with a round (smooth or corrugated, see FIG. 1A and FIG. 13, respectively) ending may be used allowing a relatively low retracting force, when compared to standard tapered balloon or a balloon with a round ending. In an embodiment, the balloon has a proximal taper cone shaped with a 15-17 degree angle, and a 15 degree round cone distal taper, having a radius of about 0.5 mm at the junction of the taper and the neck. If a corrugated ending is implemented, the corrugated portion may have corrugations having a generally triangular cross-section (see, for example the corrugated part 170I of the balloon 170 of FIG. 13). However, other different types of corrugations may also be used.

The moving member 18 may be manufactured from a metal wire or tube, such as stainless steel, Nitinol, and/or from any suitable polymer, having a diameter generally in the range of 0.2-2 mm, preferably about 0.5 mm, and length generally in the range of 50-150 mm, preferably about 100 mm. The distal portion of moving member 18 is attached to the distal portion of the slidable internal tube 13 by any suitable attaching method such as but not limited to gluing bonding embedding and the like. Most preferably, the distal portion of the moving member 18 may be embedded into the wall of internal tube 13 thereby enhancing its rigidity and the grip provided therewith. The aperture 19 is adapted to allow conveniently moving the moving member 18 therethrough while providing suitable sealing of the hollow interior of hollow shaft 6, thereby preventing leakage of inflation fluid therefrom. It is noted that the moving member 18 may be a single member or several members, such as but not limited to several wires (not shown).

In operation, the inflation fluid is preferably a saline or a saline mixed with radio-opaque solution in different ratios. A syringe pump, or other suitable inflation pumps or indeflator devices, as commonly used in the field, may be used for introducing the inflation fluid into the system. The pressure in the system in its various states typically varies between low pressure (vacuum) and up to 25 atmospheres.

While different types of over-pressure (discharge) valves may be employed, over-pressure valve 16 is preferably implemented by an annular element having an axial slender passage passing therein. In such implementation over-pressure valve 16 is manufactured from an elastomer type of material, such as PVC by an injection molding process. Its outer diameter is generally in the range of 2-6 mm, preferably about 4 mm, and its slender passage is designed to expand whenever a pressure gradient of about 4 bar develops between its ends.

Optionally, in accordance with an embodiment of the rapid exchange catheter, a piston-like member 18c is attached to the moving member 18 (or formed as a contiguous part thereof). An elongated cylindrical portion 6b is formed in the proximal part of the hollow shaft 6. The piston-like member 18c is movably disposed within the cylindrical portion 6b as illustrated in FIG. 1E. The piston-like member 18c allows for a syringe like action of the moving member 18 when the member 18 is retracted proximally, causing the piston-like member 18c to retract proximally within the cylindrical portion 6b allowing the accommodating of a sufficient amount of inflation fluid ejected from the inflated balloon 11a during retraction of the piston-like member 18c. This accommodation of inflation fluid in the space created by the proximal moving of the piston-like member 18c prevents substantial pressure increase in the catheter and in the balloon 11a during retraction of the member 18 and the intussuscepting of the balloon 11a. Thus, the combination of the piston-like member 18c and the cylindrical portion 6b, forms a syringe-like structure operative as a pressure adjusting mechanism. This pressure adjusting or pressure regulating mechanism advantageously reduces or prevents substantial pressure changes within the catheter internal space and within the balloon when the balloon is intussuscepting.

Turning to FIG. 1C, when the balloon 11a is in the inflated state and the member 18 is pulled proximally, balloon 11a folds as its distal end collapses and invaginates internally within the balloon 11a forming a cavity 3a defined by the inwardly folded distal portions of the balloon 11a. The volume encompassed by the cavity 3a may be enlarged by (partially or entirely) deflating the balloon 11a in this folded state (as illustrated in FIG. 1D). Such partial or full deflation of the balloon 11a may result in filling the enlarged cavity 3a with samples of particulate matter and/or debris from the treatment site.

It is noted that when the distal portion 11d of the balloon 11a collapses and the balloon 11a folds, the distal portion 11d is internalized such that their formerly externally facing walls now form part of the walls defining the internal cavity 3a. Therefore, any debris that was attached or adhered to the surface of corrugated portion 11e will be entrapped in the cavity 3a of the inflated balloon 11a by being carried into the cavity 3a by the internally moving outer surface of the intussuscepting parts of the inflated balloon 11a and trapped within the cavity 3a of the inflated balloon 11a and (after deflation of the balloon) of the deflated balloon 11a.

In accordance with an embodiment of the catheter of the present application, using a balloon which has higher resistance to folding at its proximal end (for example, the proximal conical part of the proximal portion 11p) relative to the resistance to folding of its distal end will ensure (or increase the probability of) preferential collapse of the distal end of the balloon. This may be achieved, inter alia, by using a balloon having a steeper taper at the distal end of the distal portion, or by using a balloon having a proximal portion with thicker walls and a distal portion with thinner walls, or by using a balloon having a corrugated distal portion and a non-corrugated proximal portion (and/or middle portion, in balloon having three part form) in which the distal corrugated portion preferentially collapses during the application of a longitudinal pulling force. It is noted that a combination of any or of all of the above means for achieving preferential collapse of the distal portion of the balloon may be used in the steeped balloons of the present application, in accordance with an embodiment of the catheters of the present application. Various different examples of such balloons with preferential collapse of a selected end are disclosed in more detail hereinafter.

The procedure for using the balloon catheter(s) of the present application may be briefly described as follows:

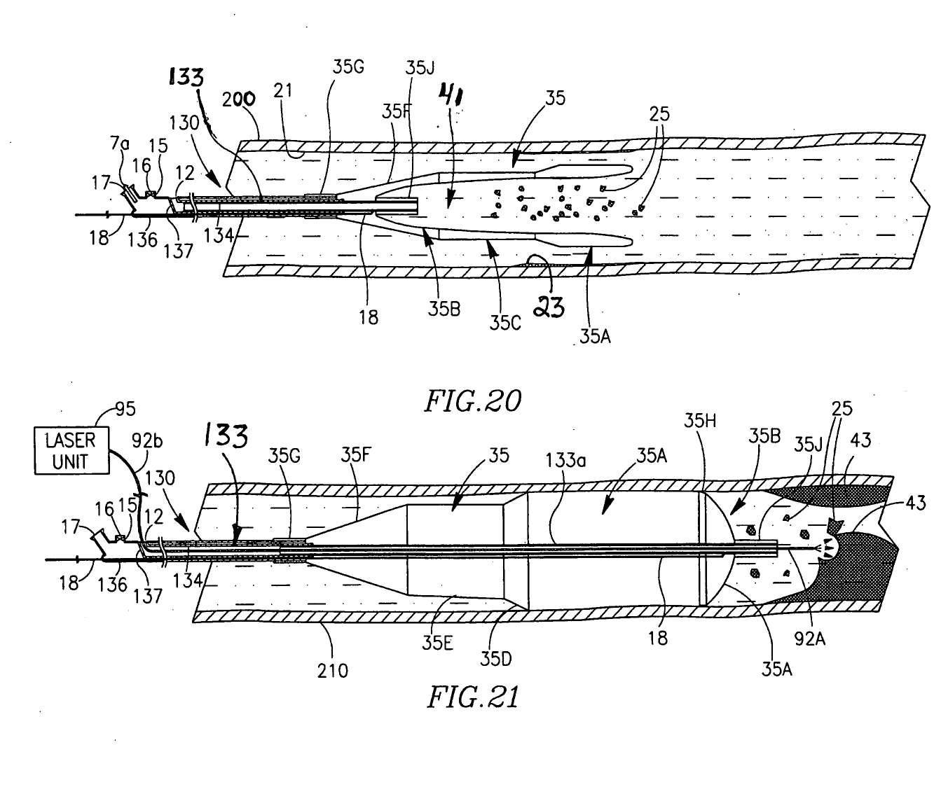

1) Inserting of catheter into the body via peripheral blood vessel by use of standard rapid exchange methods, as are well known in the art (a guide-wire may be used for the insertion, such as for example the guide-wire 5 of FIG. 1A) until the balloon reaches the target area to be treated.
2) Inflating the balloon 11a by injecting inflation fluid via fluid port 17 and the inner lumen of outer shaft 6, as demonstrated by fluid inflation arrows 7a in FIG. 1A. The pressure inside balloon 11a may be in general about 1-25 Atmospheres, preferably about 6 Atmospheres. The inflating of the balloon may already constitute treatment of the target region, such as in the case where the balloon (in the non-inflated state is disposed within a stenosed or occluded or diseased target region and the inflation of the balloon expands the balloon causing compaction and/or opening of the occluded region by the cylindrical part of the distal portion 11d. In this state, with the balloon catheter 10 inflated and firmly anchored at the treatment site, the inner lumens of inner tube 14 and of slidable internal tube 13 may now be utilized for operating at the treated site with different interventional tools (not shown) inserted through the lateral port 12 into the lumen of the inner tube 14 and the slidable internal tube, as may be required. It is noted that while the portion 11d of the balloon 11a may be placed within the region of the plaque or atheromatous occlusion and may used to treat the plaque by pushing the plaque towards the walls of the blood vessel (not shown) to open a larger passage within the atheromatous portion of the blood vessel, other different treatment methods are also possible, in which the proximal portion 11d is not used as a plaque treating or plaque pushing means, but is used as an anchoring portion of the corrugated balloon 11a enabling firm anchoring of the catheter 10 to the walls of the blood vessel which in turn allows other different plaque treating devices (not shown) to be inserted into the lumen of the inner tube 14 (after withdrawal of the guide-wire 5, or alternatively without withdrawal of the guide-wire if the device(s) are included in the guide-wire 5) for treating the plaque. In such alternative treatment methods, the balloon is typically positioned within the blood vessel at a site proximal to the position of the plaque or occluded region, and treatment is performed by an additional treating device (such as, but not limited to, a rotablator burr, a mechanical cutting device, a laser device such as an excimer laser or other laser for performing ELCA or other types of laser based atherectomies, a radiofrequency angioplasty device, an ultrasonic ablator device, and the like) inserted into the lumen of the inner tube 14. However, some procedures (for example angioplasty) may be completed, or may be near completion, once the balloon 11a reaches its fully inflated state. A specific example of such a treated method is disclosed in detail and illustrated in FIG. 20 hereinafter.
3) If required, a sample or other liquid or solid matter (for example fluids, secretions, and/or debris) may be collected from the treatment site, by pulling the moving member 18 proximally to retract the slidable internal tube 13 proximally, as demonstrated by arrow 8a in FIG. 1B. During retraction of the slidable internal tube 13 by the operator, the distal end of the balloon 11a collapses and its outer surface portions are folded inwardly over the distal end of slidable internal tube 13 and thereafter over itself as further portions of the balloon collapse, as illustrated in FIG. 1C.
4) Retraction of slidable internal tube 13 and the resulting inward folding of the balloon 11a shorten the overall length of the inflated balloon 11a which reduces the volume of inflated balloon 11a. Consequently, the pressure of the inflating fluids increases, which may result in a considerable pressure increase in balloon 11 and inner lumen of outer shaft 6. Whenever the pressure in balloon 11a and in the inner lumen of outer shaft 6 reaches a certain set-point or threshold value (for example, 5-20 atmospheres) inflation fluids are discharged via over-pressure valve 16, as shown by arrows 7b in FIG. 1B, such that the pressure in balloon 11a and inner lumen of outer shaft 6 remains within a predetermined pressure range (such as, for example 5-20 atmospheres, but other pressure threshold values are possible). Turning to FIG. 1E, another exemplary option for preventing substantial pressure increase within the catheter during intussuscepting of the balloon 11a in the catheter embodiment illustrated in FIG. 1E, is by proximally moving the piston-like member 18c of the member 18 so it acts similar to a syringe action and accommodates the inflation fluid ejected from the balloon during the proximal pulling of the member 18, as disclosed in detail with respect to FIG. 1E. During this step, the operator may determine via a graduated scale (not shown) provided on moving member 18 (or on the piston-like member 18c) the length of the part of the inner tube 14 that has been retracted and in this way determine when to stop the retraction of inner tube 14.
5) Subsequently, the balloon 11a is deflated by withdrawal of inflation fluids via fluid port 17. As a result, the pressure inside the balloon 11a and the inner lumen of outer tube 6 is substantially decreased, and the balloon 11a is deflated. The reduction in the volume of balloon 11a results in enlargement of the distal cavity 3a with the result of further drawing and capturing of additional particulate matter an/or debris from the lumen of the blood vessel into the enlarged cavity 3a. This second capturing action may occur in addition to the first debris capturing step which occurs earlier during the intussuscepting of the inflated balloon 11a.
6) The operator may then retract the balloon catheter 10 proximally such that at least some of the fluid/secretion and/or debris confined within the cavity 3a are withdrawn with the balloon catheter 10 (not shown in the figures). The debris, objects or samples captured may be easily collected when the entire length of balloon catheter 10 is withdrawn from the body of the treated subject, by pushing the inner tube 14 distally and unfolding the folded portions of balloon 11a, thus restoring the deflated state of balloon 11a (shown in FIG. 1A).

Reference is now made to FIG. 1F which is a schematic cross sectional view of part of a rapid exchange catheter in which the overpressure valve 16 is replaced by a compliant member 9. This arrangement is usable as a pressure adjusting mechanism in accordance with another embodiment of the catheter systems of the present application. The catheter 110 of FIG. 1F is similar in construction and operation to the catheter 10 of FIG. 1A, except that the over-pressure valve 16 of FIG. 1A is replaced by a compliant member 9 such as (but not limited to) an inflatable and expandable balloon made from latex or from any other suitable expandable compliant material. The compliant member 9 is sealingly attached to the outlet 15 to seal the outlet 15. In this embodiment, the outlet 15 is in fluidic communication with the lumen of the inflatable balloon 11a. When the balloon 11a of the catheter is intussuscepted while it is in the inflated state (by pulling the moving member 18 proximally), the compliant member 9 may expand to accommodate some of the inflating fluid ejected from the balloon 11a thus relieving some of the over-pressure in the lumen of the balloon 11a.

Moreover, in accordance with yet another embodiment of the catheters of the present application, a portion or portions of the hollow outer shaft 6, thereof, may be made inflatable or expandable or compliant, such that over-pressure conditions may be at least partially resolved by the expansion of the compliant portion(s) hollow outer shaft 6. Such an expandable or inflatable portion of the outer conduit is capable of expanding when over-pressure conditions occur in the lumen of the balloon to at least partially relieve the over-pressure in said lumen.

Reference is now made to FIG. 1G which is a schematic cross sectional view of part of a rapid exchange catheter in which the overpressure valve 16 is replaced by a stopcock and a hydraulic accumulator.

The catheter 120 is similar to the catheter 10, except that in the catheter 120 includes a closable stopcock 42 and a hydraulic accumulator 52, instead of the overpressure valve 15 (of catheter 10). The hydraulic accumulator 52 of the catheter 120 is fluidically connectable to the fluid port 16 via the closable stopcock 42. The stopcock 42 may be closed to fluidically isolate the hydraulic accumulator 52 from the fluid filled space within the hollow shaft 6. The stopcock 42 may also be opened to fluidically connect the hydraulic accumulator 52 to the fluid filled space within the hollow shaft 6. It is noted that the stopcock 42 is optional and is not obligatory to the operation of the catheter 120. Therefore, in accordance with an alternative embodiment of the catheter 120, the catheter 120 does not include a stopcock and the hydraulic accumulator 52 is directly fluidically connected to the fluid filled space within the hollow shaft 6 by the fluid port 16.

The hydraulic accumulator 52 is designed to accommodate fluid ejected from the balloon 11a during intussuscepting thereof. The structure and operating of hydraulic accumulators is well known in the art, is not the subject of the present application and is therefore not described in detail in the present application.

Briefly, a hydraulic accumulator is designed to accommodate excess fluid while preventing excessive increase in the pressure in a fluidic system to which it is fluidically connected. This may be achieved by several different designs such as but not limited to hydraulic accumulators using a bladder, hydraulic accumulators using a moving piston disposed in a compressible gas chamber, hydraulic accumulators using a chamber with a spring loaded piston therein, and other types of hydraulic accumulators as is well known in the art. It is noted that in FIG. 1G, the hydraulic accumulator 52 is represented by the conventional engineering symbol labeled 52 and is not drawn to scale as the structure and operation of such hydraulic accumulators is well known in the art.

When inflation fluid is ejected from the balloon 11a of the catheter 120 into the hydraulic accumulator 52, the pressure increases somewhat, but as the volume available within the hydraulic accumulator 52 is relatively large in comparison with the volume of fluid ejected from the balloon 11a during intussuscepting thereof, the pressure increase within the catheter 120 is attenuated and is not large enough to prevent the intussuscepting of the balloon 11a. The dimensions, accommodated volume and other characteristics of the hydraulic accumulator 52, such as the maximal pressure developed in the catheter after the balloon 11a has been fully intussuscepted may be selected depending, inter alia, on the dimensions of the balloon 11a, the volume ejected from the balloon 11a during intussuscepting, the balloon's inflation pressure, and other design considerations.

In operation of the catheter 120, an indeflator (not shown) may be fluidically connected to the fluid port 17 of the catheter 120 and the stopcock 42 is closed. The catheter 120 may then be inserted into the body and the balloon 11a (not shown in FIG. 1G) is placed at or near the region to be treated as disclosed in detail hereinabove. The balloon 11a may then be inflated by injecting inflation fluid under pressure using the indeflator. After treatment of the target region is performed, the stopcock 42 may be opened and the moving member 18 may be pulled proximally to cause intussuscepting of the balloon 11a and disclosed hereinabove. The pressure accumulator 52 attenuates the pressure increase within the catheter 120 and the intussuscepted balloon 11a as explained hereinabove (and may also cause partial deflation of the balloon 11a due to flowing of some of the fluid ejected from the balloon 11a into the hydraulic accumulator 52. After intussuscepting of the balloon 11a is completed, the balloon 11a may be further deflated through the fluid port 17 by using the indeflator or by disconnecting the indeflator from the fluid port 17. The catheter 120 may then be withdrawn from the body as described hereinabove.

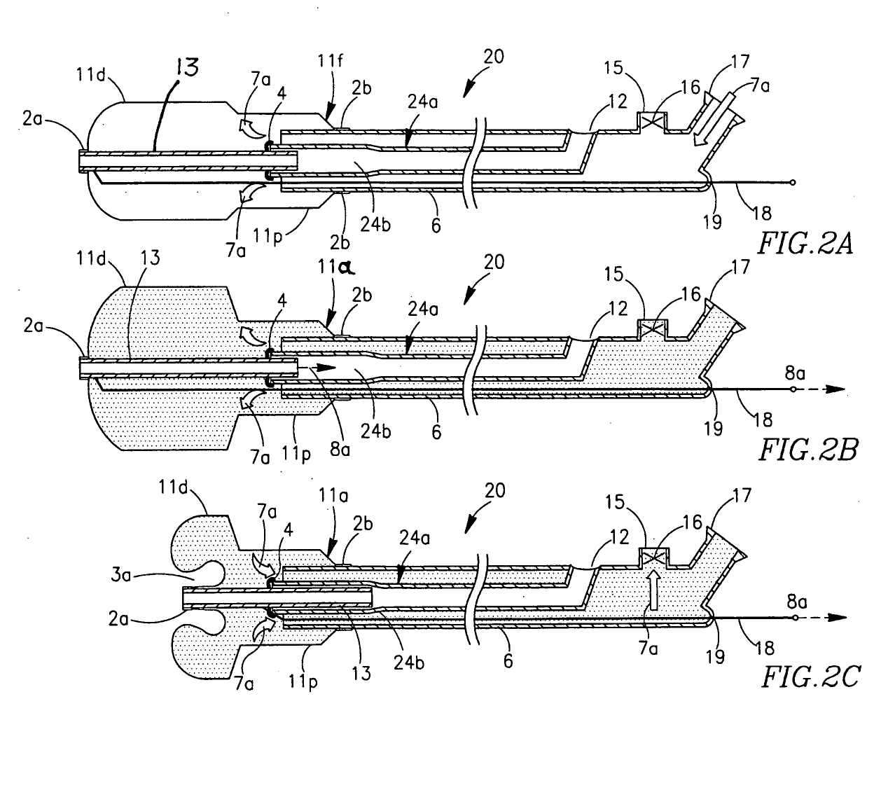

Reference is now made to FIGS. 2A-2C which are schematic cross sectional views illustrating a rapid exchange catheter having a stepped balloon in which the diameter of the distal portion of a fixed inner tube is adapted to receive an internal slidable tube, in accordance with another embodiment of the rapid exchange catheter of the present application.

The rapid exchange catheter 20 includes an inner tube 24a having a non-uniform cross-section. The diameter of a distal portion 24b of the inner tube 24a is adapted to receive an internal slidable tube 13. In this embodiment, the diameter of the distal portion 24b of the inner tube 24a is larger than the diameter of the proximal portion thereof. The internal slidable tube 13 is configured to tightly fit into the proximal portion 24b to seal its distal opening and prevent leakage of inflation fluid thereinto. Alternatively or additionally, sealing may be achieved by a gasket 4 attached to the distal portion 24b of the inner tube 24a such that a distal portion of the gasket 4 is pressed against an annular portion of the outer surface of slidable internal tube 13. The internal slidable tube 13 and the proximal portion of inner tube 24a may be manufactured to have lumens having the same inner diameter, thereby forming a substantially uniform inner passage therealong, particularly when internal slidable tube 13 is advanced all the way into the distal portion 24b.

The structure and geometrical dimensions of the components of catheter 20 are much the same as those components designated by the same reference numerals which were described above with reference to FIGS. 1A to 1C. In addition, the construction of the catheter tubes such that they are able to withstand the axially-directed stretching and buckling forces in this, and in all subsequent embodiments, are as described hereinabove, in connection with the first-described embodiment. The balloon 11a may be inflated by inflation fluid (as schematically illustrated by the arrow 7a) introduced via the inflation fluid port 17, and the length of the catheter 20 and the shape and volume of balloon 11a may be manipulated by moving the member 18 distally or proximally, as illustrated in FIGS. 2A to 2C.

The operation of the catheter 20 is similar to the operation of the catheter 10 as disclosed in detail hereinabove. Briefly, after insertion of the catheter into the body (preferably using a guide-wire, such as, for example, the guide-wire 5 disclosed in FIG. 1A), the balloon 11a may be positioned at or near the target to be treated and the balloon may be inflated with inflation fluid through the fluid port 17. After or during treatment of the target region (by using either the distal portion 11d of the balloon 11a as the treating part or any other treating device(s) or instrument(s) inserted through the lateral port 12 or by using both the balloon 11a and one or more treating and/or diagnostic devices or instruments as disclosed in detail hereinabove for the catheter 10), the balloon 11a is inflated as disclosed hereinabove.

When the moving member 18 is pulled proximally, the internal slidable tube 13 attached thereto slides proximally within the portion 24b of the inner tube 24a causing the intussuscepting of the inflated balloon 11a forming a cavity 3a and internalizing at least part of the distal portion 11d with some of the debris (not shown) which may have adhered thereto into the cavity 3a.

The inner tube 24a may be manufactured by an extrusion and laser cutting process from a plastomeric or metallic type of material, preferably from nylon, PET or stainless steel, as disclosed in detail hereinabove. The diameter of the distal portion of inner tube 24a is generally in the range of 0.3-2 mm, preferably about 0.5 mm, and the diameter of slidable internal tube 13 is adapted to provide tight fitting and the necessary sealing of distal opening of inner tube 24a when the internal tube is inserted therein. However, other different dimensions may also be used depending inter alia, on the, length and wall thickness of the inner tube 24a and of the internal slidable tube 13, the particular medical application and on engineering and other manufacturing considerations.

It is noted that while in the embodiments of the catheters illustrated in FIG. 1A and FIG. 2A, the slidable tube 13 is inserted into the distal part of the inner tubes 14 and 24a, respectively, and slides therewithin, this is not obligatory and the catheters of the present application may be constructed such that the slidable tube 13 slides over the inner tube 14 by suitably modifying the diameters of these components.

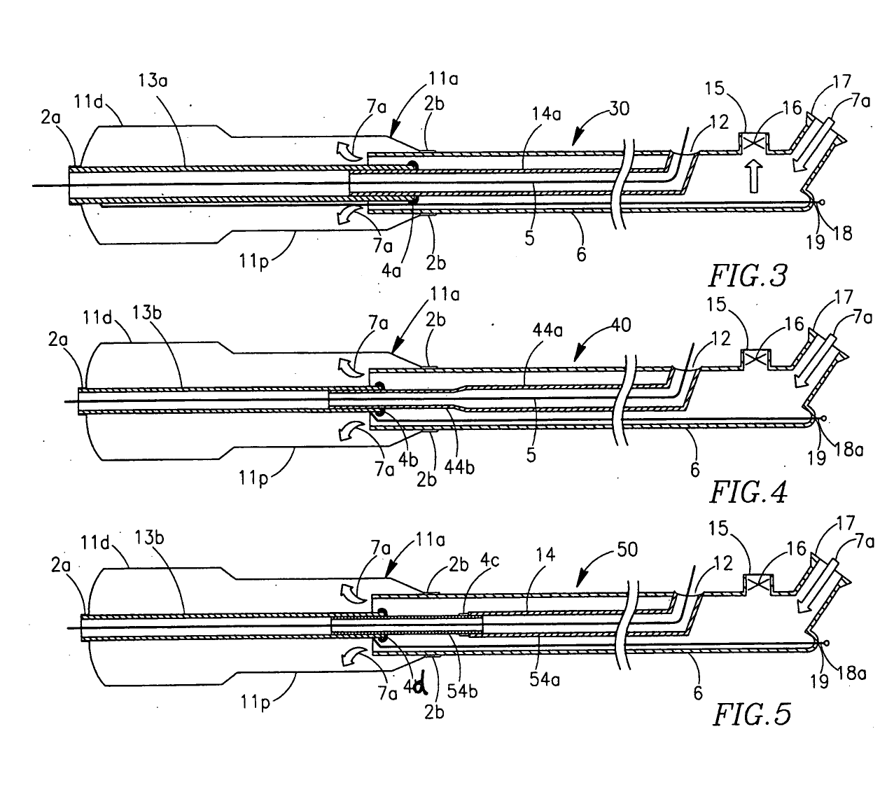

Reference is now made to FIG. 3 which is a schematic cross sectional view illustrating a rapid exchange catheter with a stepped balloon, in which an external slidable tube slides over the distal portion of a fixed inner tube, in accordance with yet another embodiment of the catheter of the present application.

In the catheter 30, an external slidable tube 13a is sealingly and slidably fitted over the distal end of an inner tube 14a. The inner diameter of the external slidable tube 13a is only slightly larger than the outer diameter of the inner tube 14a to ensure smooth sliding and a sufficient sealing to prevent leaking of inflation fluid under pressure.

In this embodiment the distal end of balloon 11a is attached to the external slidable tube 13a at distal attachment region 2a provided around the outer surface of a distal portion of the external slidable tube 13a. The diameter of the external slidable tube 13a is made slightly larger than the diameter of inner tube 14a. The external slidable tube 13a is designed to tightly fit over the outer surface of the proximal section of inner tube 14a and to thereby seal its distal opening and prevent leakage of inflation fluid thereinto. Alternatively or additionally, sealing may be achieved by gasket 4a attached to the proximal end portion of external slidable tube 13a such that a proximal portion thereof is pressed against an annular portion of the outer surface of inner tube 14a.

Using the external slidable tube 13a in catheter 30 permits the attachment of a relatively short moving member 18a to the proximal portion of the slidable tube 13a. Alternatively or additionally, the distal portion of the moving member 18a may be embedded into the wall of external slidable tube 13a along its longitudinal length, thereby enhancing its rigidity and the grip provided therewith.

The structure, geometrical dimensions of elements of catheter 30 designated by the same numerals, and the method of manipulating its length and balloon volume and shape, are much the same as those elements and manipulating method which were previously described hereinabove and therefore, for the sake of brevity, the elements will not be further discussed at this point.

Reference is now made to FIG. 4 which is a cross sectional view illustrating a rapid exchange catheter in which the diameter of the distal portion of the inner tube is adapted to be received within an external slidable tube according to yet another embodiment of the catheter of the present application. In the catheter 40, the diameter of the distal portion 44b of an inner tube 44a is adapted to be received in an external slidable tube 13b. In this embodiment, the distal end of balloon 11a is attached to the slidable external tube 13b at distal attachment region 2a provided around the outer surface of a distal portion of the slidable external tube 13b. The diameter of distal portion 44b of inner tube 44a is made relatively smaller than the diameter of the proximal portion thereof. The distal portion 44b of the inner tube 44 is constructed such that the external slidable tube 13b tightly fits over the proximal portion 44b and seals its distal opening and prevent leakage of inflation fluid thereinto. Alternatively or additionally, sealing may be achieved by gasket 4b attached to the proximal end of External slidable tube 13b such that a proximal portion thereof is pressed against an annular portion of the distal portion 44b of inner tube 44a.

The external slidable tube 13b of catheter 40 also allows attachment of a relatively short moving member 18a to the proximal portion of the slidable tube 13b. Alternatively or additionally, the distal portion of the moving member 18a may be embedded into the wall of external slidable tube 13b along its longitudinal length, thereby enhancing its rigidity and the grip provided therewith.

The structure, geometrical dimensions of elements of catheter 40 designated by the same numerals, and the method of manipulating of its length and balloon's volume and shape, are much the same as those elements and the manipulating method which were previously described hereinabove and therefore will not be further discussed here. The inner tube 44a may be manufactured by an extrusion and laser cutting process from a plastomeric or metallic type of material, preferably from nylon or stainless steel. The diameter of the distal portion 44b of inner tube 44a is generally in the range of 0.3-2 mm, preferably about 0.5 mm, and the diameter of external slidable tube 13b is adapted to provide tight fitting and the necessary sealing of distal opening of inner tube 44a when the external tube is mounted thereover. However, other different larger or smaller diameters may be used, depending inter alia, on the, length and wall thickness of the inner tube 44 and of the internal slidable tube 13b, the particular medical application and on engineering and other manufacturing considerations.

FIG. 5 is a cross sectional view illustrating a rapid exchange catheter in which the distal part of the inner tube includes a fixed inner tube on which an external slidable tube is mounted, according to yet another embodiment of the catheter of the present application. In the catheter 50, an external slidable tube 13b is slidably mounted over an inner tube 54b protruding distally through a distal opening of fixed inner tube Ma of catheter 50. In this embodiment, the distal end of balloon 11a is attached to the slidable external tube 13b at distal attachment region 2a provided around the outer surface of a distal portion of the slidable external tube 13b. A proximal end portion of the inner tube 54b is fitted into the distal opening of the fixed inner tube 54a, such that it seals the distal opening and most of its longitudinal length protrudes distally therefrom into the hollow interior of the hollow shaft 6. The diameter of external slidable tube 13b is adapted to tightly fit over the external surface of inner tube 54b, sealing its distal opening while allowing it to be easily moved distally or proximally thereon by the operator.

A sealant 4c (such as, but not limited to, a silicon based sealant, a gasket, or the like) may be applied to the proximal end of inner tube 54b in order to provide enhanced sealing of the distal opening of fixed inner tube 54a. Sealing of the distal opening of inner tube 54b may be achieved by an annular gasket 4d attached to the proximal end of external slidable tube 13b such that a proximal portion thereof is pressed against an annular portion of the outer surface of inner tube 54b.

The gasket 4d can be made of a flexible material such as silicone or polyurethane. Alternatively, the gasket 4d may be implemented by an added lubricant such as mineral oil or silicone oil which improves the sliding between the tubes. The sealing may be further increased by increasing the pressure in the balloon.

It should be noted that the tubes 54a and 54b may be fixed tubes such that the tube 54a is fixed to the shaft 6 and tube 54b is fixed to the tube 54a (by the sealant 4c), such that the slidable tube 13b can slide over the fixed tube 54b. Alternatively, the tubes 54a and 13b may be fixed tubes such that the tube 54a is fixed to the shaft 6 and the tube 13b is attached to the distal margin of the balloon 11a (at the attachment region 2a), such that the tube 54b can slide into both tubes.

The structure, geometrical dimensions of elements of catheter 50 designated by the same numerals, and the method of manipulating of its length and balloon's volume and shape, are much the same to those elements and manipulating method which were previously described hereinabove and therefore will not be discussed here, for the sake of brevity. Fixed inner tube 54a and external slidable tube 13b may be manufactured by an extrusion and laser cutting process from a plastomeric or metallic type of material, preferably from nylon or flexible metal. Their diameters are adapted to provide tight fitting and the necessary sealing of distal openings of fixed inner tube 54a and of inner tube 54b.

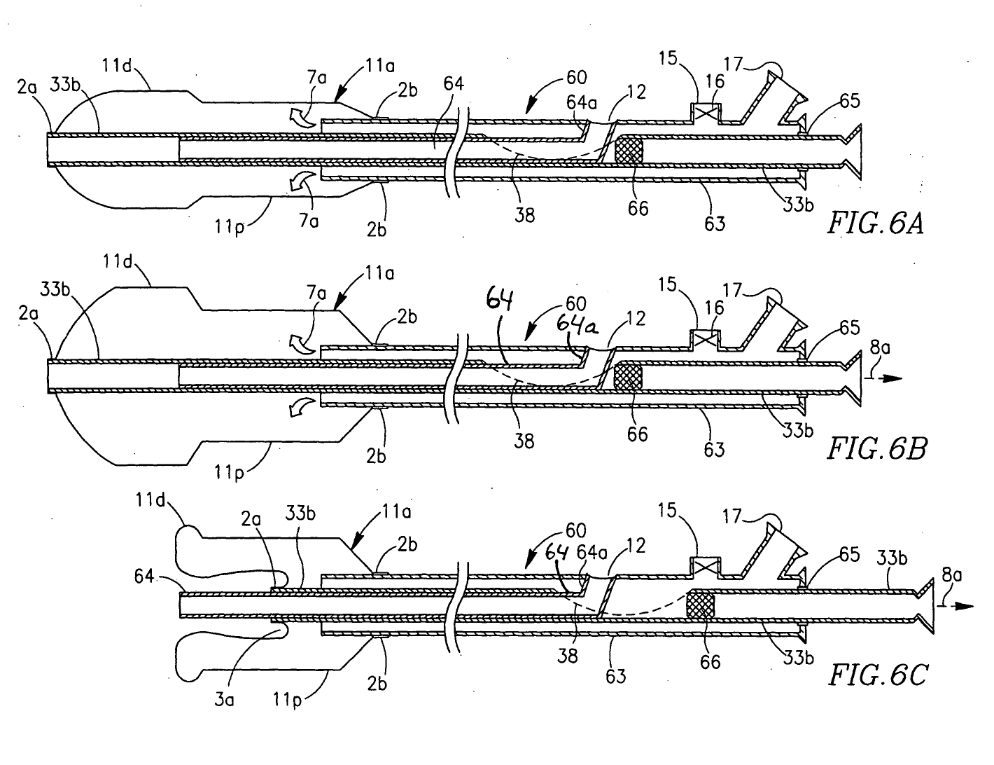

Reference is now made to FIGS. 6A to 6C which are schematic cross sectional views illustrating a rapid exchange catheter having a fixed inner tube which is encompassed by a slidable intermediate tube, according to yet another embodiment of the catheter of the present application. In the catheter 60 (FIG. 6A), the inner tube 64 of catheter 60 is encompassed in a slidable intermediate tube 33b. Both the inner tube 64 and the slidable intermediate tube 33b are disposed within a catheter hollow shaft 63. In this embodiment, the distal end of balloon 11a is attached to the slidable intermediate tube 33b at distal attachment region 2a provided around the outer surface of a distal portion of the slidable intermediate tube 33b. A longitudinal opening 38 is provided on an upper side of the slidable intermediate tube 33b. The inner tube 64 has an angled portion 64a which protrudes upwardly through the longitudinal opening 38 towards the upper side of the hollow shaft 6 at a location in which the angled portion 64a it is fixedly and sealingly attached to the shaft 6. Access to the lumen of the inner tube 64 is provided by the lateral port 12.

During a procedure, the balloon 11a may be inflated by pressurized fluid (designated by arrows 7a in FIG. 6A) provided via inflation fluid port 17. As illustrated in FIG. 6B, pressurized inflation fluid passes through the hollow interior of hollow shaft 63 into the internal space of balloon 11a. The catheter 60 and the balloon 11a in the inflated state are illustrated in FIG. 6B. The proximal portion of the intermediate tube 33b between the longitudinal opening 38 and the proximal end of the intermediate tube 33b may be sealed by a sealant 66 in order to prevent entry of inflation fluids thereinto (the sealant may be any suitable sealant known in the art including but not limited to a silicon based polymeric material, such as Silgard®, and the like). Whenever the pressure in the balloon 11a and hollow interior of hollow shaft 63 is greater than a predetermined threshold value, a portion of the inflation fluids is discharged via over-pressure valve 16 installed in a valve outlet 15.

The proximal portion of intermediate tube 33b protrudes proximally via proximal opening 65 provided at the proximal end of shaft 63. The proximal opening 65 is designed to conveniently allow the sliding of the intermediate tube 33b therethrough while providing suitable sealing thereof and preventing leakage of inflation fluid therefrom. Manipulation of the catheter length and its balloon shape and volume are performed by sliding the intermediate tube 33b proximally or distally relative to the catheter shaft 63.

For example, after inflating the balloon 11a, the operator may pull the proximal portion of intermediate tube 33b (in the direction represented by the arrow 8a in FIG. 6B), causing the distal portion of the balloon 11a to collapse and fold inwardly forming the cavity 3a, as illustrated in FIG. 6C. The longitudinal opening 38 is constructed to allow the sliding of the intermediate tube 33b proximally into a state in which the attachment point 2a reaches the distal end of the shaft 63, and on the other hand, to allow sufficient distal sliding of intermediate tube 33b in order to enable distending of the full length of the balloon 11a.

The intermediate tube 33b may be manufactured by extrusion or laser cutting processes, from a plastomer or metallic type of material such as Nylon®, Teflon®, or flexible stainless steel or any other suitable material as disclosed herein. The diameters of the inner tube 64 and of intermediate tube 33b are adapted to allow insertion of the inner tube 64 into the lumen of the intermediate tube 33b while providing suitable sealing thereof and preventing leakage of inflation fluids thereinto. For example, the intermediate tube 33b may have an inner diameter of about 0.8 mm and the outer diameter of the inner tube 64 may be of about 0.78 mm. The intermediate tube 33b may be manufactured by an extrusion process in which the internal diameter has an appropriate tolerance to fit over the outer diameter of inner tube 64. The inner tube 64 and the intermediate tube 33b are assembled together such that angled portion 64a is located in the longitudinal opening 38 of intermediate tube 33b. Thereafter the tubes 64 and 33b may be inserted into the hollow shaft 63 and the lateral port 12 may be formed by suitably sealingly and fixedly attaching or gluing or welding the open end of the angled portion 64a to a suitable opening (not shown) preformed within the hollow shaft 63.

It should be noted that the intermediate tube 33b is not necessarily a complete tube. While the distal portion of intermediate tube 33b should be of a tubular shape, its proximal portion may have other cross-sectional shapes such as, but not limited to a semi-lunar shape (not shown). Alternatively, the proximal portion of intermediate tube 33b may be implemented by a wire attached to its distal portion and exiting the catheter 60 via the proximal opening 65.

Reference is now made to FIGS. 7A to 7B which are schematic cross sectional views illustrating a rapid exchange catheter having a movable inner tube attached to an external slidable sealing sleeve, in accordance with yet another embodiment of the catheter of the present application. In the catheter 70, an inner tube 74 is made movable by attaching it to an external slidable sealing sleeve 79. In this embodiment, the distal end of the balloon 11a is attached to the inner tube 74 at a distal attachment region 2a provided around the outer surface of a distal portion of the inner tube 74. The structure, geometrical dimensions of elements of catheter 70 designated by the same reference numerals, and the method of manipulating its length and balloon's volume and shape, are much the same as those elements and manipulating method which were previously described hereinabove and therefore will not be further discussed herein, for the sake of brevity.

The catheter 70 includes a hollow outer shaft 76 having an over-pressure valve 16 and the discharge valve outlet 15 which are constructed and operative as disclosed hereinabove for the outer shaft 6 of the catheter 10 of FIG. 1A. A fluid port 27 is disposed at the proximal part of the shaft 76, and is constructed and operative as disclosed for the fluid port 17 of FIG. 1A.

As with previous embodiments of the catheters of the present application, the inner tube 74 is disposed in the hollow interior of a catheter's hollow outer shaft 76 and an angled portion 37 thereof (or, alternatively, a curved portion thereof) comprising a lateral port 12 protrudes outwardly therefrom. A lateral opening 9 is provided on the hollow outer shaft 76 from which the angled portion 37 of the inner tube 74 protrudes outwardly from the hollow shaft 76. The lateral opening 9 is sealed by an external sealing sleeve 79 movably mounted over the outer surface of the hollow outer shaft 76. The sealing sleeve 79 tightly fits over the outer surface of the hollow outer shaft 76 and seals the lateral opening 9 and the attachment area between the sealing sleeve 79 and the angled portion 37 of the inner tube 74. Moreover, the sealing sleeve 79 is made slidable to allow its movements distally and proximally within the limits imposed by the lateral opening 9 in the shaft 76.

In this way a movable inner tube 74 is obtained. The operator may inflate (as schematically designated by arrows 7a in FIG. 7A) the balloon 11a through the fluid port 27 and may move the inner tube 74 distally or proximally by sliding the sealing sleeve 79 over the hollow shaft 76. Additionally or alternatively, a moving member 48 may be attached to the inner tube 74. The moving member 48 may be attached to a proximal portion of inner tube 74 (preferably, but not obligatorily, to the angled portion 37) and a proximal portion of the moving member 48 is made accessible to an operator via a proximal opening 75 provided at the proximal end of hollow shaft 76. The proximal opening 75 is constructed to allow conveniently sliding the moving member 48 therethrough while providing suitable sealing thereof and preventing leakage of inflation fluid.

The dimensions of the lateral opening 9 and its position along the shaft 76 are adapted to allow moving of the inner tube 74 proximally into a state in which the attachment point 2a reaches the distal end of the hollow shaft 76, and on the other hand, to allow sufficient distal movement of the inner tube 74 in order to enable distending of the balloon 11a to its full length.

The sealing sleeve 79 may be manufactured by an extrusion and laser cutting process from a plastomer or metallic type of material, preferably from Nylon®, Teflon®, flexible stainless steel and the like. The sealing and attachment of the sealing sleeve 79 and the angled portion 37 of the inner tube 74 is preferably performed by bonding these parts together by thermo-bonding or any other adhesive method such that they can move together. The diameter of the sealing sleeve 79 is adjusted according to the geometrical dimensions of hollow shaft 76. For example, if the outer diameter of the hollow shaft is about 1.2 mm then the diameter of the sealing sleeve may have an internal diameter of about 1.22 mm. However, these values are given by way of example only, and other different larger or smaller diameters may be used, depending inter alia, on the length and wall thickness of the hollow outer shaft 76 and of the external sealing sleeve 79, the particular medical application and on engineering and other manufacturing considerations.

FIG. 7C is a schematic cross sectional view illustrating part of a rapid exchange catheter having a movable inner tube attached to an internal slidable sealing sleeve, in accordance with still another embodiment of the catheter of the present application.

In the catheter 70a, an internal sealing sleeve 77 is disposed within the hollow interior of a hollow shaft 76a. In this implementation, the internal sealing sleeve 77 is pressed against the inner surface of the wall of the hollow shaft 76a near the region of a lateral opening 9a formed in the hollow shaft 76a, providing suitable sealing of the lateral opening 9a. As in the catheter (FIG. 7A), an angled portion 37 of an inner tube 74 protrudes outwardly via the internal sealing sleeve 77. The internal lumen of the inner tube 74 may be accessed by the operator via a lateral port 12 (for insertion of a guide wire and/or other instrument(s) as required). The sealing and attachment of the internal sealing sleeve 77 and the angled portion 37 of the inner tube 74 may be obtained using the same means described above with reference to catheter 70.

The internal sealing sleeve 77 may be manufactured by an extrusion and laser cutting process from a plastomeric or metallic type of material, preferably from Nylon®, Teflon®, flexible stainless steel, and the like. The sealing and attachment of the internal sealing sleeve 77 and the angled portion 37 of the inner tube 74 is preferably obtained in a similar manner as was explained hereinabove. The outer diameter of the sealing sleeve 77 is adjusted according to the inner diameter of the hollow shaft 76a. For example, if the inner diameter of the hollow shaft 76a is about 1.0 mm then the outer diameter of the inner sealing sleeve 77 may be about 0.98 mm. However, these values are given by way of example only, and other different larger or smaller diameters may be used, depending inter alia, on the length and wall thickness of the hollow outer shaft 76a and of the sealing sleeve 77, the particular medical application and on engineering and other manufacturing considerations.

All of the above mentioned parameters and dimensions of all the catheters disclosed herein and their components and parts, are given by way of example only, and may be changed in accordance with the differing requirements of the various embodiments of the present invention. Thus, the above mentioned parameters should not be construed as limiting the scope of the present invention in any way. In addition, it is to be appreciated that the different tubes, balloons, shafts, and other members, described herein may be constructed in different shapes (e.g. having oval, square etc. form in plan view) and sizes and from different materials than those exemplified in the preceding description.

It should be noted that the different balloon catheter embodiments of the invention which were described hereinabove are preferably implemented with different types of balloons enabling folding of the distal portion of the balloon. However, in accordance with additional embodiments of the catheters of the present application it may be possible to use balloons adapted for undergoing collapse of the proximal portion of the balloon as disclosed in detail in FIGS. 1D-1E of PCT publication WO/2007/042935.

Reference is now made to FIG. 8 which is a schematic side view of a sleeve-like element useful for making an expandable balloon having a stepped structure in accordance with one embodiment of the balloons of the present application. It is noted that while the balloon 35 of FIG. 8 is shown without a catheter or catheter system attached to it for better understanding of its structure, the balloon 35 (and any of the other balloons and sleeve-like elements disclosed hereinafter may be suitably attached to any of the catheter or catheter systems disclosed in the present application including, but not limited to, the catheters 10, 20, 30, 40, 50, 60, 70, 70a, 100, 110 and 120 of the present application).

The stepped balloon 35 of FIG. 8 is preferably a flexible resilient sleeve that includes a plaque treating portion 35A and two (non-plaque treating) side portions 35B and 35C. In the specific (and non-limiting) embodiment of balloon 35 illustrated in FIG. 8, the plaque treating portion 35A is shaped as a cylinder, and the balloon side portion 35C includes a frusto-conical portion 35D, a cylindrical portion 35E, a frusto-conical portion 35F and a cylindrical portion 35G. The cylindrical portion 35G is the proximal margin of the balloon 35.

It is noted that the side portion 35C is configured such that the diameters of the cylindrical portion 35E and the frusto-conical portion 35F are substantially smaller than the diameter of the plaque treating portion 35A. The side portion 35B of the balloon 35 includes a frusto-conical portion 35H, a truncated dome-like portion 35I and a cylindrical portion 35J. The cylindrical portion 35J is the distal margin of the balloon 35.

Preferably the balloon 35 is made from Nylon or another suitable biocompatible material, as is known in the art, such as, but not limited to, PET, PA12 (for example Grilamid® L25, L55 and the like), PA11, PABA, Polyether block amides (such as for example, PEBAX® 7233, 7033, 6333), various types of Grilflex® (such as, for example, ELG 6260), and the like. However, any other suitable material known in the art and suitable for fabrication of catheter balloons may be used in implementing the balloons of the present application.

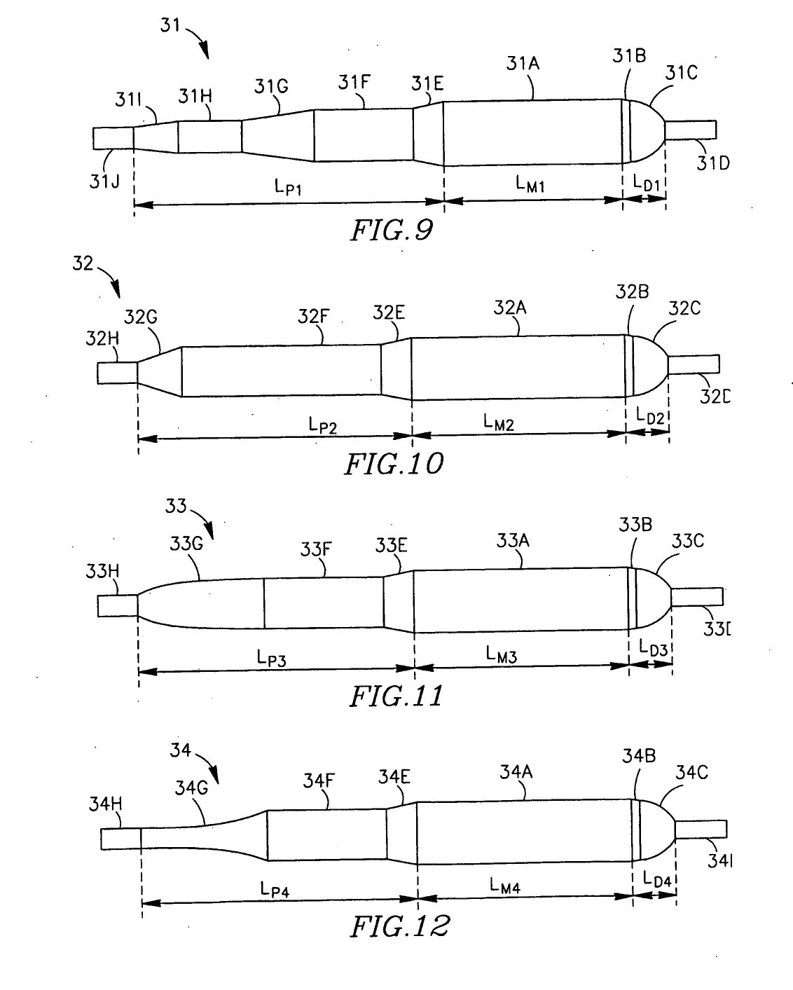

Reference is now made to FIGS. 9-12 which are schematic sideviews and cross-sectional views, illustrating various different possible embodiments of sleeve-like elements usable as inflatable balloons in the catheters and catheter systems of the present application.

In FIG. 9, the balloon 31 includes a middle portion 31A, a proximal side portion comprising contiguous portions 31E, 31F, 31G, 31H, 31I and 31J, and a distal side portion comprising contiguous portions 31B, 31C and 31D. The portions 31A, 31F, 31H 31J and 31D are cylindrical portions. The diameter of the middle portion 31A is larger than the diameters of portions 31F, 31H 31J and 31D. The diameter of portion 31J (which may be attachable to the tip of the outer shaft 6 of the catheter 10 of FIG. 1A, if balloon 31 is used instead of the balloon 11a) is larger than the diameter of portion 31D (which may be attachable to the tip of the inner tube 13 of the catheter 10 of FIG. 1A, if balloon 31 is used instead of the balloon 11a). The portions 31B, 31E, 31G and 31I are frusto-conical portions. Portion 31C is a rounded truncated (truncated dome-like) portion.

The length L_{P1} of the portions 31I, 31H, 31G, 31F and 31E is preferably larger than the length L_{M1} of the portion 31A. However, even more preferably, the length L_{P1} is larger than L_{M1}+L_{D1} (wherein L_{D1} is the combined length of the portions 31B and 31C.

In FIG. 10, the balloon 32 includes a middle portion 32A, a proximal side portion comprising contiguous portions 32E, 32F, 32G and 32H, and a distal side portion comprising contiguous portions 31B, 31C and 31D.

The portions 32A, 32F, 32H and 32D are cylindrical portions. The diameter of the middle portion 32A is larger than the diameters of portions 32A, 32F, 32H and 32D. The diameter of portion 32H (which may be attachable to the tip of the outer shaft 6 of the catheter 10 of FIG. 1A, if balloon 32 is used instead of the balloon 11a) is larger than the diameter of portion 32D (which may be attachable to the tip of the inner tube 13 of the catheter 10 of FIG. 1A, if balloon 32 is used instead of the balloon 11a). The portions 32B, 32G and 32E are frusto-conical portions. Portion 32C is a rounded truncated (truncated dome-like) portion.

The length L_{P2} of the portions 32G, 32F and 32E is preferably larger than the length L_{M2} of the portion 32A. However, even more preferably, the length L_{P2} is larger than L_{M2}+L_{D2} (wherein L_{D2} is the combined length of the portions 32B and 32C.

In FIG. 11, the balloon 33 includes a middle portion 33A, a proximal side portion comprising contiguous portions 33E, 33F, 33G and 33H, and a distal side portion comprising contiguous portions 33B, 33C and 33D. The portions 33A, 33F, 33H and 33D are cylindrical portions. The diameter of the middle portion 33A is larger than the diameters of portions 33A, 33F, 33H and 33D. The diameter of portion 33H (which may be attachable to the tip of the outer shaft 6 of the catheter 10 of FIG. 1A, if balloon 33 is used instead of the balloon 11a) is larger than the diameter of portion 33D (which may be attachable to the tip of the inner tube 13 of the catheter 10 of FIG. 1A, if balloon 33 is used instead of the balloon 11a). The portions 33B and 33E are frusto-conical portions. Portion 33G is a convex tapering portion and portion 33C is a rounded truncated (truncated dome-like) portion.

The length L_{P3} of the portions 33G, 33F and 33E is preferably larger than the length L_{M3} of the portion 33A. However, even more preferably, the length L_{P3} is larger than L_{M3}+L_{D3} (wherein L_{D3} is the combined length of the portions 33B and 33C.

In FIG. 12, the balloon 34 includes a middle portion 34A, a proximal side portion comprising contiguous portions 34E, 34F, 34G and 34H, and a distal side portion comprising contiguous portions 34B, 34C and 34D. The portions 34A, 34F, 34H and 34D are cylindrical portions. The diameter of the middle portion 34A is larger than the diameters of portions 34A, 34F, 34H and 34D. The diameter of portion 34H (which may be attachable to the tip of the outer shaft 6 of the catheter 10 of FIG. 1A, if balloon 34 is used instead of the balloon 11a) is larger than the diameter of portion 34D (which may be attachable to the tip of the inner tube 13 of the catheter 10 of FIG. 1A, if balloon 34 is used instead of the balloon 11a) The portions 34B and 34E are frusto-conical portions. Portion 34G is a concave tapering portion and portion 34C is a rounded truncated (truncated dome-like) portion.

The length L_{P4} of the portions 34G, 34F and 34E is preferably larger than the length L_{M4} of the portion 34A. However, even more preferably, the length L_{P4} is larger than L_{M4}+L_{D4} (wherein L_{D4} is the combined length of the portions 34B and 34C.

As may be seen from the above disclosed non-limiting examples, the proximal side of the stepped balloons of the present application may include any desired combination of portions, including but not limited to, cylindrical, frusto-conical, concave tapering, convex tapering, and other desired forms as long as their largest diameters are smaller than the diameter of the middle portion of the expandable balloon. Thus, the diameter of the portion(s) of the balloon which do not come in contact with the body passage walls (such as, for example, the walls of a blood vessel) which are also referred to herein as the non-treating portion(s) is smaller than the diameter of the portion of the balloon which comes in contact with the walls of the blood vessel (which are also referred to herein as the treating portion), in order to reduce possible damage during inflation (expanding) of the balloon to treat the target region.

Additionally, the inflatable distal portion of the balloons of the catheters of the present application may include one or more dome-like portions, truncated dome-like portions, conical portions, frusto-conical portions, corrugated dome-like portions, corrugated conical portions, corrugated frusto-conical portions, corrugated truncated dome-like portions and combinations of the above.

Preferably, the summed length of all the portions of the proximal side of the balloon (excluding the length of the most proximal portion used for attachment of the proximal side of the balloon to the outer shaft 6 of the catheter, such as, for example the portions 35G, 31J, 32H, 33H and 34H of FIGS. 8, 9, 10, 11 and 12, respectively) is equal to or greater than the length of the middle portion of the balloons (such as the middle portions 35A, 31A 32A, 33A and 34A, respectively).

Even more preferably, the combined length of all the portions of the proximal side portion of the balloon (excluding the length of the most proximal portion used for attachment of the proximal side of the balloon to the outer shaft 6 of the catheter, such as, for example the portions 35G, 31J, 32H, 33H and 34H of FIGS. 8, 9, 10, 11 and 12, respectively) is equal to or greater than the sum of the length all the portions of the distal side of the balloon (excluding the length of the most distal portion used for attachment of the distal side of the balloon to the inner tube 17 of the catheter, such as, for example, the portions 35J of FIGS. 8 and 31D, 32D, 33D and 34D of FIGS. 9, 10, 11 and 12, respectively) and the length of the middle portion of the balloon (such as the middle portions 35A, 31A 32A, 33A and 34A, of FIGS. 8, 9, 10, 11 and 12, respectively).

These length relationships were found to advantageously provide a sufficiently large volume of the cavity 41 (of FIG. 19 hereinafter) for trapping and containing fluids and/or debris without unnecessarily increasing the length of the balloon's (distal or middle) portion which is in contact with blood vessel walls during the period of maximal balloon expansion, while still satisfactorily maintaining a good seal between the blood vessel walls and a portion of the outer surface of the balloon after the completion of intussuscepting of the balloon (as represented in FIG. 19) and before the balloon is inflated.

However, it is noted that in a preferred embodiment of the present catheters, the dimensions of the balloon may be such that the entire distal portion of the balloon may be infolded and internalized within the cavity formed in the intussuscepting balloon, such that parts which have the larger diameter are fully internalized within the cavity of the portion having the smaller diameter. This may occur by transversal and/or longitudinal folding and crumpling of the portion having the larger diameter such that it fits within the cavity formed in the portion having the smaller diameter.

Reference is now made to FIGS. 13-14 which are schematic cross-sectional diagrams illustrating two different embodiments of corrugated stepped tapering sleeve-like elements suitable for implementing catheters having a corrugated stepped tapering intussusceptible balloons in accordance with additional embodiments of the sleeve-like elements and balloon catheters of the present application.

Turning to FIG. 13, the corrugated stepped and tapering sleeve like element 170 includes a middle portion 170A, a corrugated proximal side portion 170B and a distal side portion 170C. The proximal side portion 170B comprises contiguous portions 170H, 170G and 170F. The middle portion 170A comprises contiguous portions 170M and 170D. The portion 170M has a curved (tapering) shape. The tapered portion 170M is not corrugated and the portion 170D is corrugated. The distal side portion 170C comprises a corrugated truncated conical portion 170I (which is contiguous with the corrugated portion 170D of the middle portion 170A) and a non-corrugated cylindrical portion 170J which comprises the distal margin of the balloon 170.

The portions 170H is cylindrical and comprises the proximal margin of the sleeve-like element 170. The sleeve-like element 170 may be used in a catheter similar to the catheter 10 of FIG. 1A by sealingly attaching the portion 170H attached to the outer shaft 6 and sealingly attaching the portion 170J to the distal end of the slidable inner tube 13, as described in detail hereinabove.

The portion 170G is a frusto-conical portion. The portion 170F is a cylindrical portion and has (in its inflated state) a diameter larger than the diameter of the portion 170H but smaller than the inflated diameter of the portion 170A. The internal diameter of the cylindrical portion 170I is smaller than the internal diameter of the cylindrical portion 170H. The corrugated structure of the portion 170I may facilitate the folding and intussuscepting of the balloon which is formed when the sleeve-like element 170 is sealingly attached to a catheter. The shape and dimensions of the corrugations 170K of the portion 170I may be similar to the shape and dimensions of the corrugations 170N of the portion 170D. However, this is not obligatory and the shape and dimensions of the corrugations 170K of the portion 170I may be different than the shape and dimensions of the corrugations 170N of the portion 170D.

The length L_{P5} of the portions 170G, 170F is preferably larger than the length L_{M5} of the portion 170A. More preferably, the length L_{P5} is larger than the combined length L_{M5}+L_{D5} (wherein L_{D5} is the length of the portion 170I which is the inflatable part of the distal portion 170C).

Turning to FIG. 14, the corrugated balloon 180 includes a middle portion 180A, a proximal side portion 180B and a distal side portion 180C. The proximal side portion 180B comprises contiguous portions 180H, 180G and 180F similar in shape to the portions 170H, 170G and 170F of FIG. 13, respectively. The distal side portion 180C includes the portions 180I and 180J. The portion 180I is a curved dome-like shaped portion. The portion 180I is cylindrical and comprises the distal margin of the sleeve-like element 180. However, the middle portion 180A comprises a curved tapering portion 180M that is not corrugated, and two contiguous corrugated portions 180D and 180P.

The corrugations of the portion 180D are symmetrical triangular corrugations and the corrugations of the portion 180P are symmetrical rounded or curved corrugations.

The length L_{P6} of the portions 180G, 180F is preferably larger than the length L_{M6} of the middle portion 180A. More preferably, the length L_{P6} is larger than the combined length L_{M6}+L_{D6} (wherein L_{D6} is the length of the portion 180I which is the inflatable part of the distal portion 180C).

It is noted that other embodiments with other mixed types of corrugations are also possible in the balloons (and sleeve-like elements) of the present application. For example, in accordance with an embodiment of the balloons of the present application the middle portion of the balloon may include three contiguous portions (not shown), a first portion with rounded corrugations, a second portion with symmetrical triangular corrugations and a third portion with saw tooth-like corrugations. Thus, many other combinations and sub-combinations of multiple corrugated portions (either contiguous or non-contiguous) with multiple different types of corrugations may be implemented in the balloons and balloon catheters of the present application.

It is noted that while in the embodiments of the balloons (and sleeve-like elements) disclosed hereinabove, the corrugated portion(s) occupied most of the longitudinal dimension of the balloon's middle portion (the portion having the largest diameter of all the balloon portions), this is by no means obligatory. Rather, only a part of the middle portion may be corrugated resulting in a partially corrugated middle portion. Similarly, other embodiments are contemplated in which the middle portion of the balloon is completely non-corrugated while the distal portion of the balloon or a part thereof is corrugated.

It is noted that while the wall thickness of the sleeve-like elements 35, 31, 32, 33, 34 170 and 180 is uniform, this is not obligatory and it is possible to use sleeve-like elements having a non-uniform wall thickness along their length to form balloons having an increased probability of preferential collapse of the distal balloon portion when the balloon is in the inflated state and the inner tube 13 is moved proximally within the outer shaft 6.

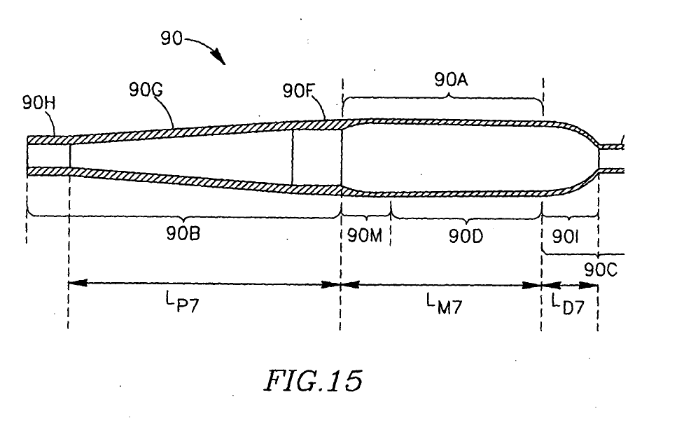

Reference is now made to FIG. 15 which is a schematic cross-sectional diagram illustrating a stepped tapering sleeve-like element having a non-uniform wall thickness usable in catheters having a stepped tapering intussusceptible balloon, in accordance with an embodiment of the balloon catheters of the present application.

In FIG. 15, a sleeve-like element 90 includes a cylindrical middle portion 90A, a proximal side portion 90B and a distal side portion 90C. The sleeve-like element 90 has a non-uniform wall thickness along its length. The proximal side portion 90B comprises contiguous portions 90H, 90G and 90F. The middle portion 90A comprises contiguous portions 90M and 90D. The portion 90M is mechanically reinforced by having a wall thickness increasing therealong in the proximal direction. Therefore, the wall thickness of the portion 90M near the distal end thereof is smaller than the wall thickness of the portion 90M near the proximal end thereof. This reinforcing advantageously increases the resistance to collapsing of the proximal portion side of the balloon 90 when the inner tube of the catheter (not shown in FIG. 15 for the sake of clarity of illustration) is pulled proximally. The portion 90D is a cylindrical portion having a uniform wall thickness equal to the wall thickness at the distal side of the portion 90M. The distal side portion 90C comprises a truncated dome-like portion 90I which is contiguous with the cylindrical portion 90D, and a cylindrical portion 90J. The wall thickness of the proximal portion 90B is uniform. The wall thickness of the proximal portion 90B is equal to the wall thickness at the proximal (and thicker) end of the portion 90M.

The wall thickness of the dome-like portion 90I of the distal portion 90C is also non-uniform. The wall thickness at the proximal end of the portion 90I is equal to the wall thickness of the portion 90D and the thickness of the walls of the portion 90I gradually diminishes in the distal direction such that the wall thickness at the distal end of the portion 90I is smaller than the wall thickness at the proximal end of the portion 90I.

The thinner wall thickness at the distal end of the distal portion 90I further increases the probability for beginning of collapse of the distal portion 90I of the balloon 90 when the inner tube (such as, for example, the slidable inner tube 13) is pulled proximally within the outer shaft 6. This combines with the reduced probability of the folding of the proximal side of the balloon 90 due to the reinforcing of the portion 90M to ensures that when the sleeve-like element 90 is attached to a catheter and a pulling force is applies by the distal tip of the inner tube to the distal portion of the balloon 90 by moving the inner tube (not shown) of the catheter in the proximal direction, as disclosed hereinabove, the distal side of the balloon 90 will fold (by collapsing) at a lower force than the force required to cause folding of the balloon at the thicker walled region of the proximal side portion 90B and the portion 90M.

The length L_{P7} of the portions 90G, 90F is preferably larger than the length L_{M7} of the middle portion 90A. More preferably, the length L_{P7} is larger than the combined length L_{M7}+L_{D7} (wherein L_{D7} is the length of the portion 90I which is the inflatable part of the distal portion 90C).

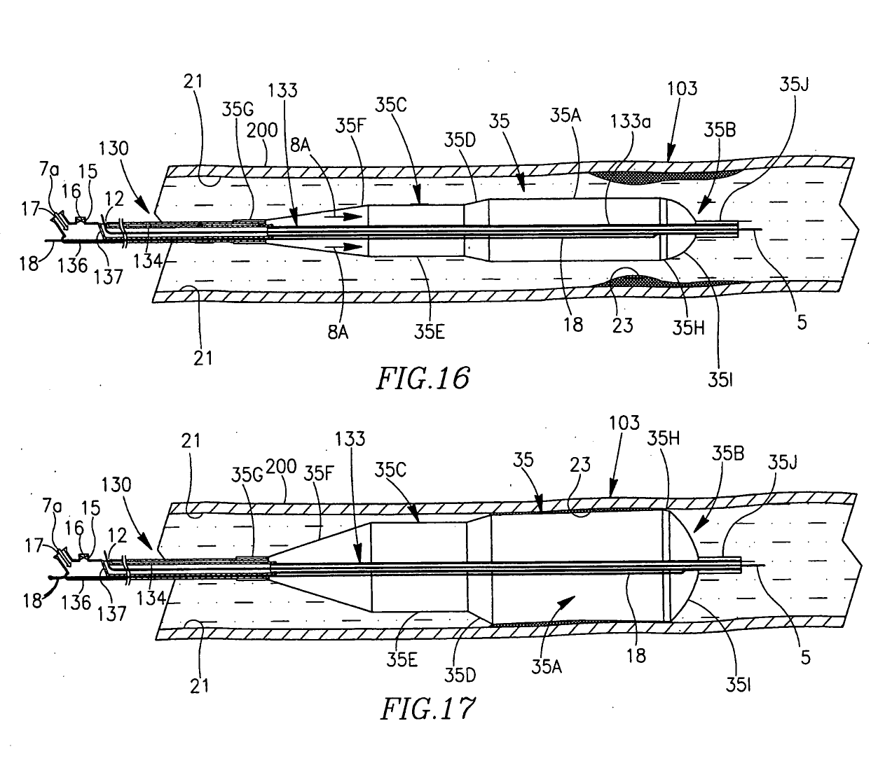

Reference is now made to FIGS. 16-20 which are schematic cross-sectional diagrams illustrating a catheter with a stepped balloon and several different steps of a method for using the catheter for treating atheromatous plaque in a blood vessel and for removing fluid and/or debris particles out of the treated blood vessel, in accordance with an embodiment of the catheter and method of use thereof of the present application.

FIG. 16 illustrates the insertion of the balloon catheter 130 of the present application to a treatment site, for example a blood vessel 200. It is noted that while the illustrations of the application use the blood vessel 200 as an example of the treated site, this is done by way of exemplary demonstration only, and other body passages may also be treated by the catheters, and catheter systems of the present application. The balloon catheter 130 includes an inner conduit 133 having a fixed inner tube 134 and a slidable inner tube 133a slidably disposed within the fixed inner tube 134. The fixed inner tube 134 is disposed within the hollow shaft 136. The proximal end of the fixed inner tube 134 comprises an entry port 12 disposed at the proximal end of an angled portion 137 which opens on the surface of the shaft 136 at the region to which the fixed inner tube 134 is attached. The shaft 136, the fixed inner tube 134 and the slidable inner tube 133a of the catheter 130 are constructed and may operate similar to the hollow shaft 6, the inner tube 14, and the slidable, internal tube 13 of the catheter 10, respectively of FIG. 1A except that some of their longitudinal dimensions may be different.

The proximal end of hollow shaft 136 further comprises a fluid port 17 for injecting/removing inflation fluids to/from inner lumen of hollow shaft 136, an over-pressure valve outlet 15 for discharging inflation fluids whenever over-pressure conditions develop in the inner lumen of hollow shaft 136, as disclosed in detail with respect to the catheter 10 of FIG. 1A, hereinabove. The balloon 35 is as disclosed in detail in FIG. 8 hereinabove and is attached to the catheter 130 as disclosed in detail hereinabove. The catheter 130 further includes a moving member 18 constructed and attached at a distal end thereof to the proximal end of the slidable inner tube 133a as disclosed in detail hereinabove for the moving member 18 of FIG. 1A).

Turning back to FIG. 16, an exemplary interventional procedure using the stepped balloon catheter 130 starts as the balloon catheter 130 is guided to the treatment site 103 within the blood vessel 200 (preferably by using the guide wire 5 as illustrated in FIG. 16). It should be clear, however, that the invention is not limited to one specific insertion method and that other appropriate and practicable catheter insertion methods known in the art (such as, but not limited to, using a guiding catheter) may also be used. The catheter 130 is advanced over the guide wire 5 until the (non-inflated) middle portion 35A is positioned within the atheromatous plaque 23 attached to the inner surface 21 of the blood vessel 200.

Turning to FIG. 17, the operator inflates the balloon 35 by injecting inflation fluids via fluid port 17 and the inner lumen of hollow shaft 136, as demonstrated by fluid inflation arrows 7a and 8a in FIGS. 16 and 17. When carrying out procedures in blood vessel 200 as demonstrated in the FIGS. 16-20, inflation fluids are preferably injected into the balloon 35 such that the circumferential sides of portion 35A of the balloon 35 are expanded and pressed against the inner surface 21 of blood vessel 200 and against the plaque 23, as illustrated in FIG. 17. The pressure inside balloon 35 in such conditions may be in general about 1-25 atmospheres, preferably about 6 Atmospheres.

It is noted that while in the embodiment of the treatment method illustrated in FIGS. 16-20 the portion 35A of the balloon is placed within the plaque 23 and is used to treat the plaque 23 by pushing the plaque 23 towards the walls of the blood vessel 200 to open a larger passage within the atheromatous portion of the blood vessel 200, other different treatment methods are also possible, in which the portion 35A is not used as a plaque treating or plaque pushing means, but is used as an anchoring portion of the balloon 35 enabling firm anchoring of the catheter 130 which in turn allows other different plaque treating devices (not shown in FIG. 16-20) to be inserted into the lumen of the inner conduit 133 (after withdrawal of the guide wire 5) for treating the plaque. In such alternative treatment methods, the portion 35A of the balloon is typically positioned within the blood vessel 200 at a site proximal to the position of the plaque, and plaque treatment is performed by an additional treating device (such as, but not limited to, a rotablator burr, a mechanical cutting device, a laser device such as an excimer laser or other laser for performing ELCA or other types of laser based atherectomies, Radiofrequency angioplasty device, an ultrasonic ablator device, and the like) inserted into the lumen of the inner conduit 133.

In this state in which the balloon catheter 130 is anchored, the inner lumen of inner conduit 133 may now be used for operating in the treated site with different interventional tools (not shown in FIGS. 16-20, but see a specific non-limiting example as illustrated in FIG. 21 hereinafter), as may be required. However, some procedures (for example angioplasty) may be completed, or may be near completion, once balloon 35 reaches its inflated state.

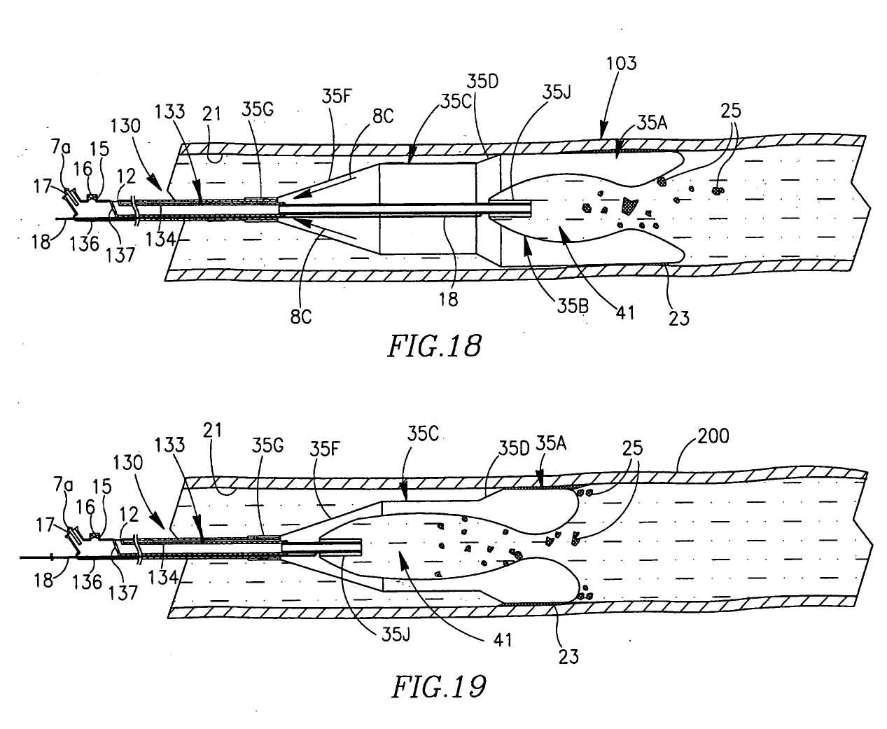

Irrespective of which particular method of plaque treatment is used, after treatment of the plaque 23 is achieved, a sample of liquid or solid matter, for example fluids, secretions, and/or debris 25 (which may possibly result from breakup of plaque 23 due to treatment steps) may be collected and removed from the treatment site by intussuscepting the balloon 35. The moving member 18 is pulled in the direction illustrated by arrow 27A in FIG. 17. The slidable inner tube 133a is then retracted proximally by the operator. During retraction of the slidable inner tube 133a, the distal portion of the balloon 35 collapses and the outer surface portions of the balloon 35 are folded inwardly over the distal tip of the slidable inner tube 133a and thereafter over itself as further portions of the balloon collapse, as illustrated in FIGS. 18-19. Thus, an internal cavity 41 is formed within the balloon 35. Debris particles 25 may be withdrawn into the cavity 41 either by being dragged inside the cavity by the formerly external surface of the balloon portion 35A to which these debris particles have become attached during the compaction of the plaque 23, or by otherwise being withdrawn into the cavity 41 due to suction of blood into the cavity during intussuscepting of the balloon 35.

The retraction of the inner tube 133a and the resulting inward folding of balloon 35, shortens the overall length of inflated balloon 35 which actually reduces the volume of inflated balloon 35. Consequently, the pressure exerted by the inflating fluids increases, resulting in a considerable pressure increase in the balloon 35 and inner lumen of hollow shaft 6. Whenever the pressure in the balloon 35 and the inner lumen of hollow shaft 136 reaches a certain set-point (such as, but not limited to 5-20 atmospheres) inflation fluids ejected from the balloon 35 flow towards the proximal side of the balloon 35 (as indicated by arrows 8C of FIG. 18), and are discharged via over-pressure valve outlet 15, such that the pressure in the balloon 35 and the inner lumen of hollow shaft 136 remains within a predetermined pressure range (e.g., 5-20 atmospheres) preventing any substantial pressure changes during the intussuscepting of the balloon 35.

After the intussuscepting of the balloon 35 has been completed, the balloon 35 is deflated by retracting inflation fluids through the fluid port 17, as indicated by arrows 8C in FIG. 20, or by simply disconnecting the indeflator or other inflation fluid supplying device from the fluid port 17. In result, the pressure inside balloon 35 and in the inner lumen of hollow shaft 136 is substantially decreased, and the intussuscepted balloon 35 is further deflated. The reduction in the pressure within the balloon 35 during deflation so results in a further increase of the volume of the cavity 41, as shown in FIGS. 18-20. This further volume increase may withdraw additional debris particles 25 into the increase volume available within the cavity 41, by suction of an additional volume of blood (with any associated debris) from the lumen of the blood vessel 200 into the expanded cavity 41.

After the intussuscepting and deflation of the balloon 35, the operator may retract (withdraw) the balloon catheter 130 proximally such that the portion of fluid/secretion and debris 25 confined within the cavity 41 are withdrawn with the balloon catheter 130 outside of the treated body (not shown in the figures). The debris, objects or samples (such as, but not limited to, the debris particles 25) collected within the cavity 41 may be easily collected when the entire length of balloon catheter 130 is withdrawn from the body of the treated subject, by moving the slidable inner tube 133a distally and unfolding the folded portions of balloon 35, thus restoring the extended state of balloon 35 (as shown in FIG. 16).

It is noted that any of the over-pressure adjusting mechanisms disclosed hereinabove with reference to any of the catheters disclosed herein may also be used in the catheter 130 as disclosed in detail

In view of the axially-directed stretching and buckling forces exerted on the inner and outer tubes during elongation and shortening of the balloon, said tubes need to be constructed such that they are able to withstand axially-directed forces in the range of between 1 and 30 Newton without undergoing deformation. In order to achieve this aim, the conduits may be constructed of a braided material or of materials having a defined molecular orientation. The approximate maximum forces that the inner and outer tubes need to withstand (for two difference size ranges of balloon inflated diameter. The inflated diameter is defined as the diameter of the balloon midsection at the balloon's nominal pressure) are as follows:

I) 2.5-4 mm diameter balloons: the tubing should withstand forces of up to 500 g; polymer tubing made of Nylon or Pebax® (a thermoplastic polyether block amide polymer) reinforced during the manufacturing process can be used.
II) 4-8 mm diameter (or larger) balloons: the tubing should withstand forces up to 2 kg. In this case it may be necessary to use a braided tube (polymer tube with metal mesh reinforcement).

Exemplary results for a representative study of the forces generated during balloon folding are presented in Example 2, of WO 2007/7004221 incorporated herein by reference in its entirety.

The hollow shaft 136 is preferably made from a biocompatible polymer type of material, such as polyurethane or nylon or PET, and may be manufactured using conventional methods, such as extrusion. The diameter of the inner lumen of hollow shaft 136 is generally in the range of 0.5-2.0 mm (millimeters), preferably about 0.7 mm, and the diameter of the fluid port 17 is generally in the range of 2-6 mm, preferably about 4 mm. The diameter of the over-pressure valve outlet 15 is generally in the range of 1-6 mm, preferably about 4 mm, and the entire length of the hollow shaft 6 is generally in the range of 100-2000 mm, preferably about 1400 mm.

The slidable inner tube 133a is preferably made from a biocompatible polymer type of material, such as polyurethane or Nylon® or PET, and it may be manufactured using conventional methods, such as extrusion. The diameter of the inner lumen of slidable inner tube 133a is generally in the range of 0.2-2.0 mm, preferably about 0.5 mm, and its entire length is generally in the range of 100-2000 mm, preferably about 1500 mm.

However, it will be appreciated by those skilled in the art that all values and dimensions of the various parts of the catheters and the values of the forces acting on the various parts as disclosed herein, are given by way of practical examples only and it may be possible to implement the catheters and balloons of the present invention by using other different values and/or value ranges of dimensions of the various parts of the catheters and/or forces to be withstood by such parts and/or different structural materials for constructing and implementing the catheters disclosed herein and any of their parts and/or components.

The balloon 35 is preferably a semi-compliant or non-compliant balloon such as the balloons manufactured by Advanced Polymers (Salem, USA) and by Interface Associates (CA). It may be manufactured using conventional methods known in the balloon catheter industry from a non-compliant type or a semi-compliant of material such as Pebax® or Nylon (preferably Nylon 12), but any other suitable material may also be used. The length of the balloon 35 is generally in the range of 10-60 mm, preferably about 20 mm. The diameter of the cylindrical portion 35A of the balloon 35 can vary from 2.0 mm to 5 mm for coronary artery applications, but may be significantly larger for use in larger blood vessels. Preferably, the balloon 35 should have a burst pressure within the range of 12-20 atmospheres. The proximal and distal edges of balloon such as the cylindrical portions 35G and 35J, respectively, of the balloon 35, are preferably sealingly attached to the outer surfaces of hollow shaft 6 and of the inner tube 17 respectively, at circumferential attachment region 7 and 6 respectively, by using a UV or thermo bonding type of adhesive such as commonly used in the art.

The shape of balloon 35 has been found by the present inventors to be important in order for the balloon to fulfill its intended functions in the presently-disclosed and claimed catheter system, namely:

i. To facilitate folding in such a way that the desired annular space is formed at the distal end of the intussuscepted balloon, by the application of the lowest possible retracting force;

ii. To present a low profile that will facilitate introduction and withdrawal of the deflated balloon into and out of the catheter system and body passage or blood vessel.

iii. To increase the volume of the cavity 41 formed within the folded (intussuscepted) balloon, while keeping the total surface of the balloon (in it's fully inflated state) that will be placed in contact with the blood vessels walls (and/or with the plaque 23) as small as possible for fulfilling its treating and/or anchoring intended function(s) and while enabling the maintaining of a seal between the blood vessel wall and at least part of the inflated portion of the balloon 35 having the largest diameter.

It appears, from modeling studies performed by the inventors, that a tapered balloon with smooth round ending folds best and has a relatively low retracting force, when compared to standard tapered balloon or a balloon with a round ending. In a particularly preferred embodiment, the balloon 35 has a proximal taper cone shaped with a 15-17 degree angle, and a 15 degree round cone distal taper, having a radius of about 0.5 mm at the junction of the taper and the neck. The results of the aforementioned modeling studies are presented in Example 2 of PCT international application published as publication number WO 2007/7004221.

Reference is now made to FIG. 21 which is a schematic cross sectional diagram illustrating a step of a method of use of the catheter system of the present application for anchoring the catheter against the walls of a blood vessel prior to the insertion of a plaque treating device through a lumen within the catheter.

In FIG. 21 the catheter 130 is shown with the balloon 35 in the fully inflated state in a blood vessel 210. The blood vessel 210 has a plaque region 43 therein. The catheter 130 is inserted into the blood vessel 210 as described hereinabove in detail with respect to FIGS. 16-20. The catheter 130 may be inserted over a guide wire 5 as shown in FIG. 16 above or by using any other catheter insertion method known in the art. The catheter 130 is suitably positioned with its distal tip at a suitable position in the vicinity of the plaque 43 and the balloon 35 is fully inflated such that its middle portion 35A firmly anchors the catheter 130 against the walls of the blood vessel 210. If the catheter 130 was guided using a guide wire, the guide wire is then withdrawn from the lumen of the inner conduit 133, through the port 12. A suitable optical fiber 92 is then inserted into the lumen of the inner conduit 133 and advanced through the lumen of the fixed inner tube 134 and the lumen of the slidable inner tube 133a until the distal tip 92A of the optical fiber 92 is positioned close to or in contact with a portion of the plaque 43. The proximal end 92B of the optical fiber 92 is optically coupled to a laser unit 95 including an excimer laser, as is known in the art. The plaque 43 may then be treated by excimer laser coronary angioplasty (ELCA) methods, as is known in the art.

Plaque particles 25 resulting from the breakup of the plaque 43 during laser treatment may then be captured and withdrawn from the body of the treated subject by withdrawing the laser fiber 92 from within the lumen of the inner conduit 133 and performing the steps for intussuscepting and deflating of the balloon 35 and withdrawing the catheter 130 out of the body of the treated subject, as illustrated and explained in detail hereinabove with respect to illustrated in FIGS. 19-20.

It is noted that while the example illustrated in FIG. 21 relates to plaque treatment using laser ablation methods, the use of the catheter systems disclosed herein is not limited to laser based plaque treating devices and methods but may rather be used in conjunction with many other types of plaque treatment devices and methods. For example, various types of mechanical plaque treating devices known in the art may be inserted into the lumen of the inner conduit 133 and used to treat the plaque 43 as is known in the art, followed by withdrawal of the mechanical plaque treating device and performing the balloon intussuscepting, deflating and catheter withdrawal steps disclosed in detail hereinabove, to effect the capture and removal of debris and/or plaque particles and/or fluids and/or secretions from the lumen of the treated blood vessel.

The plaque treating devices which may be inserted into the lumen of inner conduit 133 may include but are not limited to, rotablator burrs, blade like rotatable devices, direction cutting wires and devices, various cutting devices useful for performing directional coronary atherectomy (DCA), devices for performing directional ELCA, devices for performing radio frequency based angioplasty, and/or microwave based angioplasty and/or thermal angioplasty, devices for performing vibrational angioplasty, devices for performing physiologic low stress angioplasty (PLOSA), or any other device for treating plaque or opening an occluded blood vessel or for treating a diseased region in a blood vessel known in the art and insertable through the lumen of the inner conduit 133 of the catheter 130 or of any other catheters of the present application.

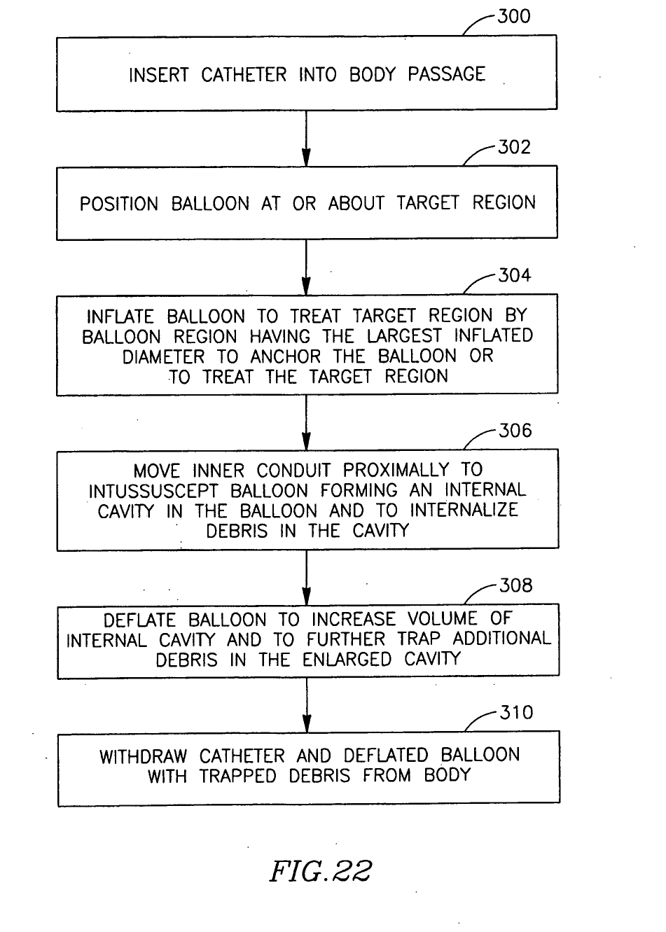

Reference is now made to FIG. 22 which is a schematic flow chart illustrating the steps of a method for using any of the catheters with stepped intussuscepting balloons of the present application for (optionally) treating a body passage and for removal of debris and/or particulate matter from a body passage, in accordance with an embodiment of the methods of the present application.

In step 300, any of the catheters disclosed herein are inserted into a body passage such as but not limited to a blood vessel, a peripheral blood vessel, a cardiac blood a pulmonary blood vessel, a renal blood vessel, a carotid blood vessel or any other blood vessel or body passage to be treated. The insertion may be performed using any type of insertion point known in the art including but not limited to, femoral artery approach, brachial artery approach, radial artery approach, carotid artery approach or any other suitable approach known in the art. The insertion may be performed by using a guide wire as is known in the art.

In step 302, the operator advances the catheter to position the balloon of the catheter near or at the target site. If the balloon is used to compact an occlusion, atheroma or plaque in a blood vessel, the balloon is positioned such that the balloon portion having the largest diameter is disposed at or within the occluded site or the atherosclerotic region target site (such as, for example, the cylindrical part of the distal portion 11d of FIG. 1A or the middle portion 35A of the balloon 35 of FIG. 8). If the balloon of the catheter is used only for anchoring the catheter, the balloon may be positioned before (proximally) to the target region, as explained with respect to FIG. 21.

In step 304, the balloon is inflated by using inflation an fluid introduced into the catheter by an indeflator or otherwise as disclosed in detail hereinabove. The inflation of the balloon may be used to anchor the balloon to the blood vessel wall in preparation to insertion of a treating device other than the balloon itself (as disclosed in detail hereinabove with respect to FIG. 21). Alternatively or additionally, the balloon may also be used for treating the occluded region or the atherosclerotic target region by compacting the plaque or by reshaping and/or distending the blood vessel wall at the occluded region (see FIGS. 16-20).

In step 306, the balloon is intussuscepted by using the moving member (such as, for example, the moving member 18, of FIG. 1A) as described hereinabove or by using any other moving mechanism (such as, but not limited to, the proximal end of the intermediate tube 33b of FIGS. 6A-6C), to collapse the end of the balloon and to form a cavity (such as for example the cavity 3a of FIG. 1C or the cavity 41 of FIGS. 18-20) within the intussuscepted balloon (preferably, but not obligatorily, the distal end of the balloon is collapsed in step 306). At this step some debris or particulate matter or secretions may be internalized or capture into the cavity (due to being dragged by the internalizing surface of the balloon during intussuscepting or by being sucked into the cavity when the cavity forms). It is noted that the debris or secretion or other particulate matter may be present in the blood vessel (or body passage) even before the catheter is inserted into the blood vessel. Such preexisting debris may also be collected and trapped in step 306. Alternatively, such preexisting debris may be collected in addition to debris resulting from the treatment (such as, for example debris resulting from deflation of the balloon within the atherosclerotic occlusion, and/or debris formed by a different treatment of the occluded region by insertion of another treating device through the lumen of the catheter, as disclosed in FIG. 21).

Furthermore, step 306 may also include (after the inflation of the balloon) the insertion of a treating device (such as, but not limited to, the laser fiber 92 of FIG. 21) through the lumen of the catheter, as disclosed in detail hereinabove. If a guide wire is present in the lumen of the catheter at this stage, the guide wire is removed from the catheter and the treating device is inserted into the catheter's lumen after the catheter is anchored.

In step 308, if a treating device has been inserted into the lumen of the catheter in step 306, the treating device is withdrawn from the catheter prior to deflating the balloon. The balloon is then deflated as disclosed in detail hereinabove. The deflating of the intussusepted balloon increases the volume of the cavity in the balloon creating additional suction of blood and possibly capturing additional debris or particulate matter carried by the additional blood withdrawn into the cavity due to increase in the cavity's volume. When the balloon is deflated, the deflated (intussuscepted) balloon contracts, releasing the anchoring of the balloon to the walls of the blood vessel. This contracting of the balloon allows the catheter to be freely moved within the blood vessel.

In step 310, the catheter is withdrawn from the body passage or the blood vessel by pulling the catheter proximally. The catheter together with any debris, and/or particulate matter and/or secretions trapped therewithin is removed outside of the body. Optionally (but not obligatorily), step 310 may also include the retrieval of any such trapped matter or debris or secretions for further examination and analysis (if desired). The trapped material(s) may be released from the cavity by pushing the moving mechanism (such as, for example, the moving member 18, of FIG. 1A or the proximal end of the intermediate tube 33b of FIGS. 6A-6C) in the distal direction. This pushing action results in longitudinal distention of the balloon until the balloon assumes its previous non-intussuscepted non-inflated state (the state of the balloon was at in steps 300 and 302 before inflation thereof). FIG. 1A illustrates a non-limiting example of the distended non-inflated balloon. This distending of the balloon releases any material or debris trapped in the balloon such that it may be collected for further observation or testing.

Turning back to FIG. 8, the inflatable proximal portion of the balloon 35 includes the frusto-conical portion 35D, the cylindrical portion 35E, and the frusto-conical portion 35F (the cylindrical portion 35G is sealingly attached to the hollow shaft 6 and is therefore not included in the inflatable proximal portion of the balloon 35), and has a length of L1. The cylindrical inflatable middle portion 35A has a length L2. The inflatable distal portion of the balloon 35 includes the frusto-conical portion 35H, and the truncated dome-like portion 35I, and has a length L3 (the cylindrical portion 35J of the side portion 35B is sealingly attached to the slidable inner tube 133a and is therefore not included in the inflatable distal portion of the balloon 35).

It is noted that similarly, for all the balloons illustrated in the drawing figures of the application, the cylindrical portions 35J, 32H, 33H, 34H, 90H, 170H and 180H are not included in the inflatable proximal portions of the balloons 31, 32, 33, 34, 90, 170 and 180, respectively as they are glued or welded or otherwise sealingly attached to the surface of the catheter parts or tubes or conduits (depending on the catheter structure being used).

Similarly, for all the balloons illustrated in the drawing figures of the application, the cylindrical portions 31D, 32D, 33D, 34D, 90J, 170J and 180J are not included in the inflatable distal portions of the balloons 31, 32, 33, 34, 90, 170 and 180, respectively, as they are glued or welded or otherwise sealingly attached to the respective part of the catheter extending beyond the distal tip of the outer shaft 6, 63 and 136.

It is further noted that the cylindrical portions 31J, 32H, 33H, 34H, 90H, 170H and 180H are also referred to as the proximal margins of the of the balloons 31, 32, 33, 34, 90, 170 and 180, respectively, throughout the specification and the claims of the present application.

Similarly, it is also noted that the cylindrical portions 31D, 32D, 33D, 34D, 90J, 170J and 180J are also referred to as the distal margins of the of the balloons 31, 32, 33, 34, 90, 170 and 180, respectively, throughout the specification and the claims of the present application.

With respect to achieving the desired function goals, detailed in item iii above, the inventors of the present application has found that it is preferable to maintain certain relationships between the various portions of the balloon 35 as follows: preferably, the length L1 should be larger than the length L2 (preferably, but not obligatorily by at least 2-3 millimeters. Even more preferably, the length L1 should be larger than the combined lengths L2+L3 (preferably, but not obligatorily by at least 2-3 millimeters). It is however noted, that while these relationships are found to be advantageous (because they ensure folding of most or of all of the external surface of the middle and/or distal portions of the balloon into the cavity formed within the proximal portion of the intussuscepted balloon), the stepped balloons disclosed herein may also be practiced with some changes from these length relationships, sacrificing full optimization of the folding and the volume of the cavity 41 in order to ensure the maintaining of a good contact between the balloon and the walls of the blood vessel 200 improving the anchoring of the balloon to the walls of the blood vessel 200 for the duration of the intussuscepting action, or to achieve other different balloon design parameters.

It is noted that the shape and number and configuration of portions of the Balloon 35 of FIGS. 8, 16-21 are given by way of example only and that other types of balloons, having different configurations, arrangements and numbers of balloon portions, may be to implemented and used in the catheters of the present application. A number of non-limiting, examples of such improved balloons are illustrated in FIGS. 1A, 2A, 3, 4, 5, 6A, and 8-15 hereinabove.

It is further noted that the balloons of the present application are not limited to the particular examples disclosed and illustrated and that various combinations of balloon features may be used such as but not limited to, tapered stepped balloons with non-uniform wall thickness and at least one corrugated portion (such as a fully or partially corrugated inflatable middle portion, and/or a fully or partially corrugated inflatable distal portion and the like).

Similarly, in balloons having a corrugated part, any type and shape of corrugations (including, but not limited to, triangular corrugations, rounded corrugations, saw tooth-like corrugations, longitudinally symmetrical corrugations, longitudinally non-symmetrical corrugations, and any combinations of corrugation types) may be used in implementing the tapered balloons of the present application.

Furthermore, in balloons having non-uniform wall thickness, any type of longitudinal wall thickness profile may be used that advantageously assists the reduction of the probability of collapse of the balloons proximal side (or of the collapse of the proximal balloon side in balloon configurations in which preferentialproximal collapse of the balloon is desired). Thus, other balloon parts may be reinforced which are different than the reinforced balloon parts illustrated in FIGS. 14-15.

Typically, in the reinforced balloons and sleeve-like elements of the present application, the ratio of the wall thickness of the thinnest part of the balloon wall to the wall thickness of the thickest part of the balloon may be in the range of 0.2-0.5. However, other ratios below or above this range may also be used depending, inter alia, on the balloon dimensions, the material used for making the balloon, the balloon's nominal inflation pressure, and other mechanical and design considerations.

It will be appreciated that the stepped balloons of the catheters and catheter systems disclosed in the present application may also be used for delivering, positioning and expanding any suitable type of stent or stents as is known in the art of balloon mediated stent deployment.

It is also noted that while most of the examples disclosed herein illustrate catheters and catheter systems particularly suitable to treating plaque in blood vessels this is not intended to limit the scope of the balloons catheters and systems to treatment of blood vessels. Rather, the balloons, catheters and systems disclosed in the present application may be used for performing various different types of treatment within bodily passages different than blood vessels and for capturing and removing solid and/or fluid materials and/or particles from within such bodily passages and withdrawing such removed materials outside the body of the treated subject.

It is also noted that while, in embodiments of the catheters which are designed for preferential collapse of the distal end of the balloon, the proximal portion of the balloons are preferably not corrugated (in order to minimize the probability of initial collapse of the proximal portion of the balloon when a proximally pulling force is applied to the balloon), it is possible to construct and use embodiments of balloon catheters including balloons having a corrugated proximal part and balloon catheters having the entire balloon being corrugated (continuously or alternatingly). For example, in accordance with other embodiments of the balloon catheters of the present application, if the balloon is made to have a corrugated proximal part or to be corrugated along the entire balloon length, the probability of the proximal collapse of the balloon during applying a force for proximally pulling of the balloon may be substantially reduced by making the walls of the proximal part of the balloon thicker than the walls of the middle and/or distal parts of the same balloon. This will enable the use of such balloons safely and effectively while allowing a greater part of the balloon to be corrugated.

It is further noted that typically (but not obligatorily) the balloon catheters of the present application may have a substantially cylindrical middle portion flanked by a distally extending portion and a proximally extending portion. The diameter of the distally extending portion typically diminishes in the distal direction and the diameter of the proximally extending portion typically diminishes in the proximal direction. The change of the diameter of the distal and/or proximal balloon portions may be gradual (as in a conical shape or dome shape but may also be non-gradual or at least partially non-gradual by diminishing abruptly (as in the form of a step or a step or an abrupt transition between a first cone angle to a steeper cone angle). Additionally the balloons of the present application may be non-linearly tapered in their proximal and/or distal portions by having outwardly or inwardly curving cross sectional shapes of the proximal and/or distal portions.

It is noted that the side portion(s) of the stepped balloons of the present application may have cylindrical and/or conical and/or frusto-conical, and/or rounded truncated dome-like and/or tapering shape(s). The side portion(s) may also have a shape which is a combination of one or more of cylindrical, conical, frusto-conical, dome-like and tapering shapes. These shapes are not intended to be limiting, and other different types of portion shapes may also be used in implementing the corrugated balloons of the present application.

The balloon catheters of the present application may use sleeve like elements having various different dimensions. Typically (but not obligatorily), the inflated diameter of the balloon may be in the range of 1.5-35 mm and the length of the balloons may be in the range of 5-300 mm. All possible combinations of balloon length and balloon diameters within these ranges may be used in implementing the balloons of the present application. In accordance with some typical non-limiting examples, a balloon with a length of 15 mm may have an inflated diameter of 3 mm and a balloon with a length of 250 mm may have an inflated diameter of 12 mm. The typical (but non-limiting) range of balloon wall thickness is 0.022-0.030 mm depending, inter alia, on the balloon dimensions and on the application. It will be appreciated by those skilled in the art that the above dimension ranges and ratios of balloon diameter to balloon length are not obligatory and that other different dimensions and ratios extending beyond the above indicated ranges may be used in implementing the catheters, depending, inter alia, on the particular application.

While it is possible for the corrugations to span the entire inflatable length of the stepped balloons, as disclosed herein, typically, in some preferred embodiments only the distal portion of the balloon is corrugated and in some other preferred embodiments, both the distal balloon portion and part of the balloon middle portion are corrugated. Typically, in these embodiments between a fifth (⅕) and a third (⅓) of the total length of the balloon are corrugated. However, shorter or longer portions of the balloon length may be corrugated, depending, inter alia, on the balloon structure and shape, the balloon's wall thickness (and/or on the balloon's wall thickness gradient in balloons with a non-uniform wall thickness), and on the particular application.

1. A rapid exchange balloon catheter comprising:
an outer conduit;
an inner conduit disposed within said outer conduit and suitable for total or partial passage over a guide-wire, said inner conduit comprises at least one movable part movably disposed within the lumen of said outer conduit, said inner conduit comprises a proximal angled portion piercing the wall of said outer conduit and a distal end extending beyond the distal end of said outer conduit;
an inflatable balloon having a proximal margin sealingly attached to the outer surface of the distal end of said outer conduit, and a distal margin sealingly attached to the outer surface of the portion of said inner conduit that extends beyond the distal end of said outer conduit, said inflatable balloon includes a first portion having a first diameter and at least a second portion having a second diameter smaller than said first diameter;
means for axially moving said at least one movable part of said inner conduit within said outer conduit;
means for the introduction of an expansion fluid into the space formed between said outer conduit and said inner conduit and therefrom into the lumen of said balloon and for the removal of said fluid from said space and from said lumen; and
means for permitting unhindered axial movement of said at least one movable part of said inner conduit within said outer conduit, such that said movement is not hindered by the passage of said angled portion of the inner conduit through said outer conduit.

2. The rapid exchange balloon catheter according to claim 1, wherein the means for axially moving comprise one or more elongated moving members, the distal end(s) thereof being attached to said at least one movable part of said inner conduit, and the proximal end(s) thereof extending beyond the proximal end of the outer conduit.

3. The rapid exchange balloon catheter according to claim 1, wherein the distal portion of said balloon is capable of intussuscepting upon proximal movement of said at least one movable part of said inner conduit in relation to said outer conduit.

4. The rapid exchange balloon catheter according to claim 1, also including means for reducing pressure changes within said space upon axial movement of said at least one movable part of said inner conduit in relation to said outer conduit.

5. The rapid exchange balloon catheter according to claim 4, wherein said means for reducing pressure changes comprises a piston-like member slidably disposed within the proximal end of the outer conduit, wherein said piston-like member is connected to said means for axially moving, such that upon operation of said means for axially moving said piston-like member is caused to move either distally or proximally, changing the volume of said outer conduit.

6. The rapid exchange catheter according to claim 1, wherein said means for permitting unhindered axial movement comprise a sealing sleeve sealingly attached to said angled portion of said inner conduit and slidably fitted around said outer conduit, such that said angled portion of said inner conduit passes firstly through an elongated aperture in the wall of said outer conduit, and secondly through a tightly sealed aperture in said sealing sleeve, such that upon axial movement of said at least one movable part of said inner conduit, said sealing sleeve is capable of preventing leaking of inflation fluid through said elongated aperture.

7. The rapid exchange catheter according to claim 1, wherein said means for permitting unhindered axial movement of the inner conduit is provided by a two-part inner conduit construction, wherein a first proximal part of said two-part inner conduit comprises a non-movable inner tube including said angled portion, and a second distal part of said two-part inner conduit comprises a slidable internal tube disposed within said non-movable inner tube.

8. The rapid exchange catheter according to claim 1, wherein said means for permitting unhindered axial movement of the inner conduit is provided by a two-part inner conduit construction, wherein a first proximal part of said two-part inner conduit comprises a non-movable inner tube including said angled portion, and a second distal part of said two-part inner conduit comprises a slidable internal tube disposed over said non-movable inner tube.

9. The rapid exchange catheter according to claim 1, wherein said means for permitting unhindered axial movement of said inner conduit is provided by a two-part inner conduit construction, wherein said two-part inner conduit comprises
a non-movable inner tube including said angled portion, and
a slidable intermediate tube movably disposed between said non movable inner tube and said outer conduit, said intermediate tube has an elongated longitudinal opening on its side through which said angled portion passes, wherein the distal end of said intermediate tube is the portion of said inner conduit that extends beyond the distal end of said outer conduit, said distal margin of said balloon is sealingly attached to the outer surface of the distal end of said intermediate tube, and wherein the proximal end of said intermediate tube sealingly passes through and extends beyond the proximal end of said outer conduit such that said means for axially moving comprises said proximal end of said intermediate tube.

10. The rapid exchange catheter according to claim 1, wherein said means for permitting unhindered axial movement of said inner conduit is provided by a three-part inner conduit construction, wherein said three-part inner conduit comprises,
a first non-movable hollow tube including said angled portion at its proximal portion and having a distal end,
a second non-movable hollow inner tube having a proximal end and a distal end, said second inner tube is sealingly disposed within said distal end of said first non-movable inner tube, and
a third slidable inner tube slidably disposed over the distal end of said second non-movable hollow tube, said third slidable inner tube has a distal end extending beyond the distal end of said outer conduit, wherein said distal margin of said balloon is attached to the outer surface of the portion of said distal end of said third inner tube extending beyond the distal end of said outer conduit.

11. The rapid exchange catheter according to claim 1, wherein said outer conduit includes a lateral opening therein, and wherein said means for permitting unhindered axial movement comprise a sealing sleeve internally disposed within said outer conduit and attached to said angled portion of said inner conduit, said sealing sleeve is sealingly fitted within said outer conduit, such that said angled portion of said inner conduit passes firstly through the wall of said sealing sleeve, and secondly through said lateral opening of said outer conduit, such that upon axial movement of said inner conduit and said sealing sleeve, said sealing sleeve is capable of preventing leaking of inflation fluid through said lateral opening.

12. The rapid exchange balloon catheter according to claim 1, wherein said inflatable balloon is characterized by having, in its inflated state, a shape which is capable of guiding the intussuscepting of the distal end thereof upon proximal movement of the at least one movable part of the inner conduit in relation to the outer conduit.

13. The balloon catheter according to claim 1 wherein said first portion comprises at least a first cylindrical portion, said at least second portion is proximal to said first portion and comprises at least a second cylindrical portion having in the inflated state a diameter smaller than the diameter of said first cylindrical portion in the inflated state.

14. The balloon catheter according to claim 13 wherein said second portion also comprises at least two frusto-conical portions flanking the distal and the proximal sides of said second cylindrical portion.

15. The balloon catheter according to claim 1 wherein said second portion comprises at least one frusto-conical portion.

16. The balloon catheter according to claim 13, wherein said second portion comprises one or more portions selected from, cylindrical portions, frusto-conical portions, concave tapering portions, convex tapering portions, and combinations thereof.

17. The balloon catheter according to claim 13 wherein the length of said second proximal portion is equal to or larger than the length of said first portion.

18. The balloon catheter according to claim 1 wherein said at least a second portion comprises a second portion proximal to said first portion and a third portion distal to said first portion, and wherein the inflated diameter of said third portion is smaller than the diameter of said first portion.

19. The balloon catheter according to claim 18 wherein the length of said second proximal portion is equal to or larger than the combined length of said first portion and said third portion.

20. The balloon catheter according to claim 1 wherein at least part of said inflatable balloon is corrugated.

21. The balloon catheter according to claim 1 wherein said balloon has a non-uniform wall thickness along its longitudinal axis.

22. The balloon catheter according to claim 21 wherein said at least a second portion is proximal to said first portion of said balloon and the wall thickness of at least part of said first portion is smaller than the wall thickness of at least part of said second portion of said balloon.

23. The balloon catheter according to claim 1, wherein said first portion of said inflatable balloon comprises one or more portions selected from dome-like portions, truncated dome-like portions, conical portions, frusto-conical portions, corrugated dome-like portions, corrugated conical portions, corrugated frusto-conical portions, corrugated truncated dome-like portions and combinations thereof.

24. The balloon catheter according to claim 1, wherein said balloon catheter also includes a pressure adjusting mechanism for preventing substantial pressure changes within said space and the lumen of said balloon upon proximal axial movement of said at least one movable part of said inner conduit in relation to said outer conduit.

25. The balloon catheter according to claim 24, wherein said pressure adjusting mechanism is selected from,
a pressure adjusting mechanism comprising a syringe-like structure disposed at the proximal end of said catheter, said syringe-like structure includes a piston-like member, said syringe-like structure is in fluidic communication with said space, said piston like member is movably disposed within said syringe-like structure and is mechanically coupled to said means for axially moving said at least one movable part of said inner conduit, such that when said movable part of said inner conduit is moved proximally the amount of inflation fluid ejected from said balloon during the intussuscepting thereof is accommodated within said syringe-like structure;
an outlet in fluidic communication with the lumen of said inflatable balloon and having an opening and a compliant member sealingly attached to said opening for at least partially relieving over-pressure in said lumen,
an over-pressure valve outlet in fluidic communication with the lumen of said inflatable balloon and an over-pressure valve disposed within said over-pressure outlet to allow discharging of fluid from said lumen when over-pressure conditions develop in said lumen,
an expandable or inflatable portion of said outer conduit, capable of expanding when over-pressure conditions occur in the lumen of said balloon to at least partially relieve the over-pressure in said lumen, and
a hydraulic accumulator configured for being controllably fluidically connected and disconnected from said space and said lumen of said balloon.

26. The balloon catheter according to claim 1, wherein said first portion of said balloon is distal to said at least a second portion and is characterized by having, in its inflated state, a distal end shape selected from the group consisting of a distal taper with a rounded distal extremity, a dome-like portion, a truncated dome-like portion, a conical portion, a frusto-conical portion, a corrugated dome-like portion, a corrugated conical portion, a corrugated frusto-conical portion, and a corrugated truncated dome-like portion.

27. A method of constructing an intussusceptible balloon rapid exchange catheter, the method comprising the steps of:
providing a catheter having an outer conduit, an inner conduit disposed within said outer conduit and suitable for total or partial passage over a guide-wire, said inner conduit comprises at least one movable part movably disposed within the lumen of said outer conduit, said inner conduit comprises a proximal angled portion piercing the wall of said outer conduit wherein a distal end of said inner conduit extends beyond the distal end of said outer conduit;
providing an inflatable balloon having a proximal margin and a distal margin, said inflatable balloon includes a first portion having a first diameter and at least a second portion having a second diameter smaller than said first diameter; and
sealingly attaching said proximal margin of said balloon to the outer surface of the distal end of said outer conduit and sealingly attaching said distal margin of said balloon to the outer surface of the portion of the inner conduit that extends beyond the distal end of said outer conduit such that the lumen of said balloon is in fluidic communication with the space defined between said outer conduit and said inner conduit, said attaching is performed such that the distal end of said balloon is capable of intussuscepting upon proximal movement of said at least one movable part of said inner conduit in relation to said outer conduit.

28. A method for collecting debris from an internal passage of a mammalian subject comprising the steps of:
a) inserting a rapid exchange balloon catheter into said internal passage, and advancing said catheter until the distal end thereof has reached a site at which it is desired to collect debris, said rapid exchange balloon catheter comprises,
an outer conduit,
an inner conduit disposed within said outer conduit and suitable for total or partial to passage over a guide-wire, said inner conduit comprises at least one movable part movably disposed within the lumen of said outer conduit, said inner conduit comprises a proximal angled portion piercing the wall of said outer conduit and a distal end extending beyond the distal end of said outer conduit,
an inflatable balloon having a proximal margin sealingly attached to the outer surface of the distal end of said outer conduit, and a distal margin sealingly attached to the outer surface of the portion of said inner conduit that extends beyond the distal end of said outer conduit, said inflatable balloon includes a first portion having a first diameter and at least a second portion having a second diameter smaller than said first diameter,
means for axially moving said at least one movable part of said inner conduit within said outer conduit,
means for the introduction of an expansion fluid into the space formed between said outer conduit and said inner conduit and therefrom into the lumen of said balloon and for the removal of said fluid from said space and from said lumen, and
means for permitting unhindered axial movement of said at least one movable part of said inner conduit within said outer conduit, such that said movement is not hindered by the passage of said angled portion of the inner conduit through said outer conduit;
b) inflating said balloon with expansion fluid;
c) moving said at least one movable part of said inner conduit in a proximal direction, such that the distal end of said first portion of said balloon collapses and said balloon intussuscepts forming a cavity for collecting said debris;
d) deflating said balloon, to increase the volume of said cavity to collect and trap additional debris within said cavity; and
e) removing the balloon catheter from said internal passage together with the entrapped debris.

29. The method according to claim 28, wherein the internal passage is a blood vessel.

30. A method for collecting debris resulting from treatment of a diseased target region of an internal passage of a mammalian subject comprising the steps of:
a) inserting a rapid exchange balloon catheter into said internal passage, said rapid exchange balloon catheter comprises an outer conduit,
an inner conduit disposed within said outer conduit and suitable for total or partial passage over a guide-wire, said inner conduit comprises at least one movable part movably disposed within the lumen of said outer conduit, said inner conduit comprises an angled portion piercing the wall of said outer conduit, wherein the distal end of said inner conduit extends beyond the distal end of said outer conduit,
an inflatable balloon having a proximal margin sealingly attached to the outer surface of the distal end of said outer conduit and a distal margin sealingly attached to the outer surface of the portion of said inner conduit that extends beyond the distal end of said outer conduit, said inflatable balloon includes a first portion having a first diameter and at least a second portion having a second diameter smaller than said first diameter,
means for axially moving said at least one movable part of said inner conduit within said outer conduit,
means for the introduction of an expansion fluid into the space formed between the inner surface of said outer conduit and the outer surface of said inner conduit and therefrom into the lumen of said balloon and for the removal of said fluid from said space, and
means for permitting unhindered axial movement of said at least one movable part of said inner conduit within said outer conduit, such that said movement is not hindered by the passage of said angled portion of said inner conduit through said outer conduit, and advancing said catheter to position said balloon within at least part of said diseased target region;
b) inflating said balloon with expansion fluid to treat at least part of said diseased target region contacted by said first portion, such that at least some of the debris formed during said inflating is attached to the outer surface of said first portion;
c) proximally moving said at least one movable part of said inner conduit of said balloon catheter, such that the distal end of said balloon collapses and said balloon intussuscepts, trapping at least some of said debris within a cavity formed therein, wherein at least part of said outer surface of said first portion is internalized within said cavity;
d) deflating said balloon, to increase the volume of said cavity and to trap additional debris therewithin; and
e) removing said balloon catheter from said internal passage, together with the entrapped debris.

31. The method according to claim 30, wherein the internal passage is a blood vessel and said diseased portion comprises an atheromatous plaque.

32. The method according to claim 30, wherein said at least second portion of said balloon is longer than said first portion of said balloon, and wherein the entire outer surface of said first portion is internalized within said cavity in said step of proximally moving.

33. A method for collecting debris from an internal passage of a mammalian subject the method comprising the steps of:
inserting a balloon catheter portion as defined in claim 1 into said internal passage, and advancing said catheter until the distal end thereof has reached a site at which it is desired to collect debris;
inflating said inflatable balloon with expansion fluid;
moving said at least one movable part of said inner conduit of said catheter in a proximal direction, for collapsing the distal end of said inflatable balloon to form an intussusceptedballoon having a cavity therein into which said debris is collected and entrapped;
deflating the intussuscepted balloon; and
removing said balloon catheter from said internal passage, together with the entrapped debris.

34. The method according to claim 33, wherein the internal passage is a blood vessel.

35. The method according to claim 33, wherein said step of moving comprises moving said at least one movable part of said inner conduit in a proximal direction to form said cavity, such that all of the outer surface of said first portion of said balloon is disposed within said cavity to increase retention of said debris.

36. The method according to claim 33, wherein said catheter includes a mechanism for reducing pressure changes within the catheter when said at least one movable part of said inner conduit is moved proximally within said outer conduit while said balloon is inflated and said fluid port is closed, and wherein said step of moving comprises moving said at least one movable part of said inner conduit in a proximal direction, for collapsing the distal end of said balloon to form a cavity within said balloon into which debris is collected and entrapped without inducing substantial pressure changes within the lumen of said balloon during the intussuscepting of said balloon.

37. The method according to claim 33, wherein said internal passage is an occluded blood vessel and wherein said step of inflating comprises inflating said balloon while said balloon is disposed near or within an atheromatous plaque of said occluded blood vessel, said inflating is performed such that at least part of said first portion of said balloon is pushed against said plaque and wherein at least some debris from said plaque adheres to the outer surface of said first portion and is internalized within said cavity formed in said step of moving.

38. A method for treating a diseased target region of an internal passage of a mammalian subject and for collecting debris from said internal passage the method comprising the steps of:
inserting a balloon catheter into said internal passage, said catheter comprises an inflatable intussusceptable balloon, said balloon comprises at least a first portion having a first diameter and at least a second portion having a second diameter smaller than said first diameter, and advancing said catheter until the distal end thereof reaches said target region;
inflating said balloon such that said first portion contacts said target region to treat said target region while said at least second portion does not contact the walls of said internal passage;
collapsing the distal end of said intussusceptable balloon while said balloon is in an inflated state to form a cavity within said balloon into which debris is collected and entrapped;
deflating the intussuscepted balloon to increase the volume of said internal cavity; and
removing said balloon catheter from said internal passage together with debris entrapped within said cavity.

39. The method according to claim 38 wherein said step of deflating comprises deflating the intussuscepted balloon to increase the volume of said internal cavity and to further trap additional debris in the increased volume of said internal cavity.

40. The method according to claim 38 wherein said step of deflating comprises deflating the intussuscepted balloon to increase the volume of said internal cavity and to create suction assisting the collecting of additional debris into said cavity.

41. The method according to claim 38 wherein said step of collapsing comprises intussuscepting said balloon such that at least some of the external surface of said first portion in internalized within said cavity and wherein at least some debris attached to said external surface is trapped within said cavity.

42. The method according to claim 38 wherein said catheter comprises a mechanism for reducing pressure changes within said catheter and wherein said step of collapsing comprises collapsing the distal end of said intussusceptable balloon while said balloon is in an inflated state to form a cavity within said balloon into which debris is collected and entrapped, without causing a substantial pressure change within said balloon during said collapsing.

43. The method according to claim 38 wherein said at least a second portion comprises a proximal second portion of said balloon, the length of said second portion is greater than the length of said first portion, and wherein said step of collapsing comprises collapsing the distal end of said intussusceptable balloon while said balloon is in an inflated state to form said cavity until the entire external surface of said first portion is completely internalized within said cavity.

44. The method according to claim 38 wherein said at least a second portion comprises a proximal second portion of said balloon, the length of said first portion is greater than the length of said first portion, and wherein said step of collapsing comprises collapsing the distal end of said intussusceptable balloon while said balloon is in an inflated state to form said cavity until at least part of the external surface of said first portion is internalized within said cavity.

45. The method according to claim 38 wherein said internal passage is a blood vessel, said target region is an occluded region of said blood vessel and at least some of said debris is formed during an opening of said occluded region by said first portion of said balloon.

46. A rapid exchange balloon catheter comprising:
an outer conduit;
an inner conduit disposed within said outer conduit and suitable for total or partial passage over a guide-wire, said inner conduit comprises at least one movable part movably disposed within the lumen of said outer conduit, said inner conduit comprises a proximal angled portion piercing the wall of said outer conduit and a distal end extending beyond the distal end of said outer conduit;
an inflatable balloon having a proximal margin sealingly attached to the outer surface of the distal end of said outer conduit, and a distal margin sealingly attached to the outer surface of the portion of said inner conduit that extends beyond the distal end of said outer conduit, said inflatable balloon includes a first portion having a first diameter and at least a second portion having a second diameter smaller than said first diameter;
a moving mechanism for axially moving said at least one movable part of said inner conduit within said outer conduit, said moving mechanism permits unhindered axial movement of said at least one movable part of said inner conduit within said outer conduit, such that said movement is not hindered by the passage of said angled portion of the inner conduit through said outer conduit; and
a fluid port for the introduction of an expansion fluid into the space formed between said outer conduit and said inner conduit and therefrom into the lumen of said balloon and for the removal of said fluid from said space and from said lumen.

47. The rapid exchange balloon catheter according to claim 46, also including a pressure regulating mechanism for reducing pressure changes within said space upon axial movement of said at least one movable part of said inner conduit in relation to said outer conduit.
